(19) United States
(12) Patent Application Publication
(10) Pub. No.: US 2015/0265691 A1
(43) Pub. Date: Sep. 24, 2015

(54) Anthrose-based compositions and related methods

(71) Applicants: Denong Wang, Palo Alto, CA (US); Pavol Kovac, Silver Spring, MD (US); Lawrence Steinman, Stanford, CA (US); Gregory Carroll, Cincinnati, OH (US); Nicholas Turro, Tenafly, NJ (US); Jeffrey Koberstein, Sturrs, CT (US); Rina Saksena, Sturrs, CT (US); Roberto Adamo, Siene (IT)

(72) Inventors: Denong Wang, Palo Alto, CA (US); Pavol Kovac, Silver Spring, MD (US); Lawrence Steinman, Stanford, CA (US); Gregory Carroll, Cincinnati, OH (US); Nicholas Turro, Tenafly, NJ (US); Jeffrey Koberstein, Sturrs, CT (US); Rina Saksena, Sturrs, CT (US); Roberto Adamo, Siene (IT)

(21) Appl. No.: 11/901,234

(22) Filed: Sep. 14, 2007

Related U.S. Application Data
(60) Provisional application No. 60/844,767, filed on Sep. 15, 2006.

(51) Int. Cl. A61K 39/07; G01N 33/68; G01N 33/569; A61K 47/48; C07K 16/12; C07K 16/42

(52) U.S. Cl. CPC A61K 39/07; C07K 16/1278; C07K 16/4233; G01N 33/56911; A61K 47/48284; A61K 47/48246; G01N 33/686; A61K 2039/6068

(57) ABSTRACT

This invention provides a vaccine comprising (i) an anthrose-containing saccharide in an amount effective to enhance immunity against Bacillus anthracis in a subject and (ii) a pharmaceutically acceptable carrier. This invention provides a vaccine comprising (i) a conjugate of an anthrose-containing saccharide in an amount effective to enhance immunity against Bacillus anthracis in a subject, wherein the anthrose-containing saccharide is conjugated to a biomolecule via a linker, and (ii) a pharmaceutically acceptable carrier. This invention provides a method for vaccinating a subject against Bacillus anthracis infection comprising administering to the subject a vaccine comprising (i) an anthrose-containing saccharide in an amount effective to enhance immunity against Bacillus anthracis in the subject and (ii) a pharmaceutically acceptable carrier, in an amount effective to stimulate production of antibodies to Bacillus anthracis spores in the subject, thereby vaccinating the subject against Bacillus anthracis.

This application claims the benefit of U.S. Provisional Application No. 60/844,767, filed Sep. 15, 2006, the contents of which are hereby incorporated by reference.

The invention described herein was made with Government support under the National Institutes of Health grant number AI064104, U.S. Army Research Laboratory grant number DA W911NF-04-1-0282, and the National Science Foundation grant numbers DMR-02-14263, IGERT-02-21589 and CHE-04-15516. Accordingly, the United States Government has certain rights in this invention.

Throughout this application, various references are cited. Disclosure of these references in their entirety is hereby incorporated by reference into this application to more fully describe the state of the art to which this invention pertains.

### BACKGROUND OF THE INVENTION

Anthrax is a fatal infectious disease caused by the gram-positive, rod shaped bacterium B. anthracis. Anthrax infection is initiated by the entry of spores into the mammalian host via intradermal inoculation, ingestion, or inhalation.[1] The most lethal form of human anthrax is the pulmonary infection caused by inhaled spores. In view of the risk of B. anthracis spores as a biological weapon of mass destruction (WMD)[2], it is necessary to achieve the capacity for the rapid and specific detection of B. anthracis spores in various conditions[3, 4]. It is also important to develop new vaccines to block the anthrax infection at its initial stage before spore germination takes place[5, 6]. In this context, identification of highly specific immunogenic targets that are displayed on the outermost surfaces of B. anthracis spores is of utmost importance.

Substantial effort has been made to study the structure and antigenic elements of the outer layers of B. anthracis spores. The mature B. anthracis spore contains a central genome-containing core compartment and three adjacent protective layers: the cortex, coat, and exosporium[?-9]. The exosporium is at the outermost surface and is fully exposed to the external environment. Morphologically, the exosporium has two distinct structures, a paracrystalline basal layer and an external hair-like nap[8]. Isolated B. anthracis exosporia contain as many as 20 protein components [9, 10]. The most prominent element is the BclA (for Bacillus collagen-like protein of anthracis) glycoprotein[11, 12].

The BclA glycoprotein displays a unique rhamnose-containing tetrasaccharide. This carbohydrate moiety is capped at its upstream end with a previously unknown sugar residue termed anthrose [2-O-methyl-4-(3-hydroxy-3-methylbutamido)-4,6-dideoxy-β-D-glucose][12]. The complete structure of this tetrasaccharide has been determined to be 2-O-methyl-4-(3-hydroxy-3-methylbutamido)-4,6-dideoxy-β-D-glucopyranosyl-(1→3)-α-L-rhamnopyranosyl-(1→3)-α-L-rhamnopyranosyl-(1→2)L-rhamnopyranose[12]. Importantly, this sugar moiety is absent on the surfaces of B. anthracis vegetative cells or the spores of other bacillus strains, including B. cereus and B. thuringiensis, the two strains phylogenetically most close to B. anthracis[12].

Surface-exposed carbohydrate moieties that are characteristic of a given microbe may serve as key biomarkers for pathogen identification, diagnosis and vaccine development. Therefore, the anthrose-containing tetrasaccharide of the BclA glycoprotein is an attractive target for immunological investigations. BclA has been confirmed to be immunodominant[13]. However, whether its carbohydrate moieties contribute to the immunogenecity of this exosporium glycoprotein remains unknown[14]. This may be due to the lack of a method for the detection of antibodies specific for the carbohydrate moieties of the glycoprotein. It has, therefore, been difficult to decipher the anti-carbohydrate specificities and anti-protein specificities, even if the corresponding antibodies were present in the sera as a result of a polyclonal antibody response to a natural infection or a vaccination using immunogenic glycoconjugates.

### SUMMARY OF THE INVENTION

This invention provides a vaccine comprising (i) an anthrose-containing saccharide in an amount effective to enhance immunity against Bacillus anthracis in a subject and (ii) a pharmaceutically acceptable carrier.

This invention provides a vaccine comprising (i) a conjugate of an anthrose-containing saccharide in an amount effective to enhance immunity against Bacillus anthracis in a subject, wherein the anthrose-containing saccharide is conjugated to a biomolecule via a linker, and (ii) a pharmaceutically acceptable carrier.

This invention provides a method for vaccinating a subject against Bacillus anthracis infection comprising administering to the subject a vaccine comprising (i) an anthrose-containing saccharide in an amount effective to enhance immunity against Bacillus anthracis in the subject and (ii) a pharmaceutically acceptable carrier, in an amount effective to stimulate production of antibodies to Bacillus anthracis spores in the subject, thereby vaccinating the subject against Bacillus anthracis.

This invention provides an isolated antibody which specifically binds to an epitope on an anthrose-containing saccharide.

This invention provides a composition of matter comprising (i) an antibody which specifically binds to an epitope on an anthrose-containing saccharide and (ii) a detectable marker, wherein the detectable marker is conjugated to the antibody.

This invention provides a composition of matter comprising (i) an antibody which specifically binds to an epitope on an anthrose-containing saccharide and (ii) an antibiotic against Bacillus anthracis, wherein the antibiotic against Bacillus anthracis is conjugated to the antibody.

This invention provides an isolated anti-idiotypic antibody which specifically binds to an antibody which specifically binds to an anthrose-containing saccharide.

This invention provides a composition of matter comprising (i) an anti-idiotypic antibody which specifically binds to an antibody which specifically binds to an anthrose-containing saccharide, and (ii) a detectable marker, wherein the detectable marker is conjugated to the antibody.

This invention provides a composition comprising (i) an antibody which specifically binds to an anthrose-containing saccharide in an amount effective to enhance immunity against Bacillus anthracis in a subject and (ii) a pharmaceutically acceptable carrier.

This invention provides a method for reducing the likelihood of a subject being infected with Bacillus anthracis, which comprises administering to the subject a prophylactically effective amount of a composition comprising (i) an antibody which specifically binds to an anthrose-containing saccharide, and (ii) a pharmaceutically acceptable carrier, thereby reducing the likelihood of a subject being infected with Bacillus anthracis.

This invention provides a vaccine comprising (i) an anti-idiotypic antibody which specifically binds to an antibody which specifically binds to an anthrose-containing saccharide in an amount effective to enhance immunity in a subject against Bacillus anthracis spores, and (ii) a pharmaceutically acceptable carrier.

This invention provides a method for vaccinating a subject against Bacillus anthracis infection comprising administering to the subject an effective amount of a vaccine comprising (i) an anti-idiotypic antibody which specifically binds to an antibody which specifically binds to an anthrose-containing saccharide in an amount effective to enhance immunity in a subject against Bacillus anthracis spores, and (ii) a pharmaceutically acceptable adjuvant.

This invention provides a method for determining whether Bacillus anthracis spores are present in a serum sample of a subject which comprises: (a) contacting the serum sample with an antibody which specifically binds to an epitope on an anthrose-containing saccharide under conditions that permit the formation of a complex between the antibody and an anthrose-containing saccharide; and (b) detecting the presence of the complex formed in step (a); wherein the presence of such complex indicates that Bacillus anthracis spores are present in the serum sample.

This invention provides a method for determining whether a subject has been exposed to Bacillus anthracis which comprises (a) contacting a serum sample from the subject with an antibody which specifically binds to an epitope on an anthrose-containing saccharide under conditions that permit the formation of a complex between the antibody and an anthrose-containing saccharide; and (b) detecting the presence of the complex formed in step (a), wherein the presence of such complex indicates that the subject has been exposed to Bacillus anthracis.

This invention provides a method for determining whether a subject is infected with Bacillus anthracis which comprises (a) contacting a serum sample from the subject with a anti-idiotypic antibody which specifically binds to an antibody which specifically binds to an anthrose-containing saccharide under conditions which permit the formation of a complex between the anti-idiotypic antibody and an antibody which specifically binds to an anthrose-containing saccharide; (b) determining the amount of complex formed in step (a); and (c) comparing the amount of complex determined in step (b) with an amount of complex correlative with that found for a subject infected with Bacillus anthracis, thereby determining whether the subject is infected with Bacillus anthracis.

This invention provides a kit for detecting the presence of Bacillus anthracis spores in a sample comprising, in separate compartments, (i) an antibody which specifically binds to an epitope of an anthrose-containing saccharide and (ii) reagents for detecting binding of the antibody to an anthrose-containing saccharide.

This invention provides a kit for detecting in a sample the presence of antibodies which bind to Bacillus anthracis spores comprising, in separate compartments, (i) an anti-idiotypic antibody which specifically binds to an antibody which specifically binds to an anthrose-containing saccharide, and (ii) reagents for detecting binding of the anti-idiotypic antibody to the antibody to which it specifically binds.

This invention provides a kit for detecting in a sample the presence of antibodies which bind to Bacillus anthracis spores comprising, in separate compartments, (i) a conjugate of an anti-idiotypic antibody which specifically binds to an antibody which specifically binds to an anthrose-containing saccharide, wherein the anti-idiotypic antibody is conjugated to a detectable marker and (ii) reagents for detecting binding of the anti-idiotypic antibody to the antibody to which it specifically binds.

### BRIEF DESCRIPTION OF THE FIGURES

FIG. 1

This Figure shows photo-generated glycan arrays for rapid identification of pathogen-specific immunogenic sugar moieties. Saccharide preparations were dissolved in saline (0.9% NaCl) at a given concentration and spotted using a high-precision robot (PIXSYS 5500C, Cartesian Technologies Irvine, Calif.) onto the phthalimide-amine monolayers (PAM)-coated slides. The printed PAM slides were subjected to UV irradiation (300 nm) for 1 hr to activate the photo-coupling of carbohydrates to the surface. Pathogen-specific anti-sera were then applied on the glycan arrays to identify potential immunogenic sugar moieties of given pathogens.

FIG. 2

This Figure shows that photo-generated glycan arrays recognize immunogenic sugar moieties of the B. anthracis spore. A panel of thirty-five mono-, oligo- and polysaccharides (See supplementary table 1) were spotted on the photo-reactive glass slides followed by UV-irradiation to induce covalent coupling of the saccharides to the array substrate. These photo-generated glycan arrays were stained with rabbit anti-B. anthracis spore polyclonal IgG antibodies (Abcam Limited, Cambridge, UK) at 10 or 20 μg/ml in the absence or presence of saccharide inhibitors (0.25 mg/ml) as specified in the Supplementary table 2). The bound rabbit IgG was revealed by a tagged anti-rabbit IgG antibody. Glycan array staining, image capturing and data-processing were performed as previously described [15, 19-22]. Images (a-f) display a portion of the stained glycan arrays: (a) no saccharide inhibitor; (b) anthrose; (c) D-glucose; (d) α-anthrose trisaccharide; (e) α-anthrose tetrasaccharide; (f) β-anthrose tetrasaccharide. The locations of surface-bound anthrose-containing saccharides that are recognized by the antibody in the absence of inhibitor are highlighted by colored boxes: White (Array location B-1), β-anthrose-trisacchride; Brown (Array location C-1), β-anthrose-tetrasaccharide; Yellow (Array location B-4), α-anthrose-tetrasaccharide. Microarray data sets are shown in Supplementary Table 2.

FIGS. 3A-3C

These Figures show epitope-mapping using glycan array and saccharide inhibition assays to identify the key elements of the anthrose-containing immunogenic sugar moieties.

(A) Glycan array-based saccharide binding assays: A histogram illustration of the antibody profile of a preparation of rabbit anti-B. anthracis spore polyclonal antibodies. The levels of IgG antibody reactivity were measured as the mean values of the ratio of fluorescence intensity over the background, which is the mean value of 112 blank spots in the same glycan array). Each histogram displays the mean intensity of triplicate detections by the glycan arrays. Saccharide Id#: 1) anthrose monosaccharide; 2) anthrose-containing disaccharide; 3) anthrose-containing trisaccharide; 4) anthrose-containing β-tetrasaccharide; 5) anthrose-containing α-tetrasaccharide; 6-33) saccharides without anthrose, including monosaccharides and a panel of rhamnose-containing oligosaccharides (See Supplementary Table 1 for structural information and Supplementary Table 3 for microarray dataset).

(B) Glycan array-based saccharide blocking assays: For each candidate saccharide, at least two glycan array assays were conducted to examine its inhibition activity. Results are illustrated as the levels of saccharide-specific IgG antibodies captured by glycan arrays in the presence or absence of a saccharide inhibitor. Each histogram represents the mean value of a specific antibody reactivity detected by triplicate saccharide arrays.

(C) ELISA-based quantitative saccharide inhibition assays. This assay was performed following the KABAT laboratory's standard as described [23]. In brief, ELISA microtiter plates (NUNC, MaxiSorp) were coated with a BSA conjugate of α-anthrose-tetrasaccharide at 5 μg/ml in 0.1M sodium bicarbonate buffer, pH 9.6, and were incubated with a preparation of rabbit anti-anthrax spore IgG (2 μg/ml) in the presence or absence of varying quantities of the sugar inhibitors. Percent inhibition was calculated as follows: % inhibition=((standard A−blank A)−(A with inhibitor−blank A))/(standard A−blank A). The half maximal inhibitory concentration (IC₅₀) values for given saccharides were calculated based on mathematical models of the linear range of the corresponding saccharide inhibition curve [23].

FIGS. 4A & 4B

These Figures show saccharide inhibition assays to identify the key elements of the anthrose-containing immunogenic sugar moieties.

(A) Glycan array-based multiplex inhibition assays. For each candidate saccharide, at lease two glycan array assays were conducted to examine its inhibition activity. Results are illustrated as the levels of saccharide-specific IgG antibodies captured by glycan arrays in the presence or absence of a saccharide inhibitor. The inhibition reactivities of saccharides are quantitatively and reversely co-related to the IgG signal detected by glycan arrays. The levels of IgG are measured as the ratio of fluorescence intensity over background signal. In the above figure, each histogram represents the mean value of a specific antibody reactivity detected by triplicate saccharide arrays. (B) Saccharide inhibition assays by ELISA. A protein conjugate of α-anthrose-tetrasaccharide was coated on ELISA plate to display the saccharide structure. Saccharide inhibitors were applied in given concentrations as specified in the figure to compete with the saccharide binding on ELISA microtiter plates. Results were plotted as the percent of inhibition (I %) at each concentration (nmol.) for a given saccharide inhibitor.

FIGS. 5A & 5B

These Figures show the immunogenicity of anthrose-α-tetra-PA conjugates.
(A) Microarray images of serum IgG antibody profiles, Left:

Pre-immunization; Right: post-immunization.

(B) A quantitative comparison of IgG reactivities pre- and post-immunization with the anti-tetra-α-PA conjugate.

FIG. 6

This figure shows microarray-based semi-quantitative analysis of antigen-specific antibodies in Balb/c serum specimens pre- and post-immunization. Sera from ten Balb/c pre- and post-immunization with Ant4-PA conjugates (5) or Rha3-PA conjugates were characterized. Microarray datasets are presented below by an overlay plot of the three comparative groups. Each dot in the bottom panel is the mean value of IgG values (Int-Bg) of a given group with a cross (x) for the serum IgG pre-immunization, a dot for the group of five, mice immunized with Ant4-PA and square for the mice immunized with Rha3-PA conjugates (5).

FIG. 7

This figure shows the carbohydrate-based dominant antigenic determinants of B. anthracis spores as identified by the photo-generated glycan arrays. The dominant antigenic determinants are the terminal and internal glycol-epitopes of anthrose tetrasaccharide.

### DETAILED DESCRIPTION OF THE INVENTION

### Terms

“Administering” may be effected or performed using any of the methods known to one skilled in the art. The methods comprise, for example, intralesional, intramuscular, subcutaneous, intravenous, intraperitoneal, liposome-mediated, transmucosal, intestinal, topical, nasal, oral, anal, ocular or otic means of delivery. The embodiments of the subject invention may be administered one time or in several courses over a period of time, and may or may not require a booster annually or after several years.

“Affixed” or “bound” shall mean attached by any means. In one embodiment, affixed shall mean attached by a covalent bond. In another embodiment, affixed shall mean attached non-covalently.

“Antibody” shall include, without limitation, (a) an immunoglobulin molecule comprising two heavy chains and two light chains and which recognizes an antigen; (b) a polyclonal or monoclonal immunoglobulin molecule; and (c) a monovalent or divalent fragment thereof. Immunoglobulin molecules may derive from any of the commonly known classes, including but not limited to IgA, secretory IgA, IgG, IgE and IgM. IgG subclasses are well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies, and fragments thereof. Antibody fragments include, without limitation, Fab fragments, Fv fragments and antigen-binding fragments. Antibodies may be human or nonhuman. Nonhuman antibodies may be humanized by recombinant methods to reduce their immunogenicity in humans.

As used herein, “humanized” describes antibodies wherein some, most or all of the amino acids outside the CDR regions are replaced with corresponding amino acids derived from human immunoglobulin molecules. Small additions, deletions, insertions, substitutions or modifications of amino acids are permissible as long as they do not abrogate the ability of the antibody to bind a given antigen. Suitable human immunoglobulin molecules include, without limitation, IgG1, IgG2, IgG3, IgG4, IgA and IgM molecules. Various publications describe how to make humanized antibodies, e.g., U.S. Pat. Nos. 4,816,567 (50), 5,225,539 (51), 5,585,089 (52) and 5,693,761 (53) and PCT International Publication No. WO 90/07861 (54).

As used herein, a “detectable marker” includes but is not limited to a radioactive label, or a calorimetric, a luminescent, or a fluorescent marker. As used herein, “labels” include radioactive isotopes, fluorescent groups and affinity moieties such as biotin that facilitate detection of the labeled peptide. Other labels and methods for attaching labels to compounds are well-known to those skilled in the art.

“Determining” whether a complex is formed can be performed according to methods well known in the art. Such methods include, but are not limited to, fluorescence, radioimmunoassay, and immunolabeling detection.

As used herein, “effective amount” means an amount in sufficient quantities to either treat the subject or prevent the subject from becoming infected with Bacillus anthracis. A person of ordinary skill in the art can perform simple titration and animal experiments to determine what amount is required to treat a subject. For example, an effective amount includes, without limitation, dosage amounts of 10-100 ug/kg, 10-200 ug/kg, 10-50 ug/kg, 40-60 ug/kg, 50-70 ug/kg, 10 ug/kg-1 mg/kg, 1 mg/kg, or 150 ug/kg.

“Immobilized” shall mean attached by any means. In one embodiment, immobilized shall mean attached by a covalent bond. In another embodiment, immobilized shall mean attached non-covalently.

As used herein, “infected with Bacillus anthracis” means that the subject has at least one cell which has been invaded by Bacillus anthracis (e.g., wherein Bacillus anthracis genetic information has been introduced into a target cell, such as by fusion of the target cell membrane with Bacillus anthracis). As used herein, “treating” includes, for example, slowing, stopping or reversing the progression of a Bacillus anthracis infection. In the preferred embodiment, “treating” means reversing the progression to the point of eliminating the infection. As used herein, “treating” also means the reduction of the number of bacterial infections, reduction of the number of infectious bacterial particles, reduction of the number of infected cells, or the amelioration of symptoms associated with Bacillus anthracis infection.

“Moiety” shall mean, unless otherwise limited, any chemical or biochemical entity. Examples of moieties include, without limitation, proteins (antibodies), nucleic acids, carbohydrates, small molecules and inorganic compounds.

“Sample”, when used in connection with the instant methods, includes, but is not limited to, any body tissue, blood, serum, plasma, cerebrospinal fluid, lung fluid, saliva, mucous, skin and sweat.

“Solid support” shall include, without limitation, chips (e.g. silicone chips), slides (e.g. glass slides), filters, plates, beads and membranes (e.g. nylon membranes). The use of these and other supports are known by one skilled in the art.

The phrase “specifically binding” refers, for example, to a binding reaction, wherein the binding of a first entity to a second entity based on complementarity between the three-dimensional structures of each occurs with a K_{D} of less than 10⁻⁵. In another embodiment, specific binding occurs with a K_{D} of less than 10⁻⁸. In a further embodiment, specific binding occurs with a K_{D} of less than 10⁻¹¹.

“Subject” shall mean any organism including, without limitation, a mouse, a rat, a dog, a guinea pig, a sheep, a cow, a pig, a chicken, a rabbit and a primate. In the preferred embodiment, the subject is a human being.

### EMBODIMENTS OF THE INVENTION

This invention provides a vaccine comprising (i) an anthrose-containing saccharide in an amount effective to enhance immunity against Bacillus anthracis in a subject and (ii) a pharmaceutically acceptable carrier. In one embodiment, the vaccine further comprises a pharmaceutically acceptable adjuvant.

The instant vaccines can optionally include a pharmaceutically acceptable carrier. As used herein, “pharmaceutically acceptable carrier” means that the carrier is compatible with the other ingredients of the formulation and is not deleterious to the recipient thereof, and encompasses any of the standard pharmaceutically accepted carriers. Such carriers include, for example, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

As used herein, “adjuvants” shall mean any agent suitable for enhancing the immunogenicity of an antigen such as protein and nucleic acid. Adjuvants suitable for use with protein-based vaccines include, but are not limited to, alum, Freund's incomplete adjuvant (FIA), Saponin, Quil A, QS21, Ribi Detox, Monophosphoryl lipid A (MPL), and nonionic block copolymers such as L-121 (Pluronic; Syntex SAF). In a preferred embodiment, the adjuvant is alum, especially in the form of a thixotropic, viscous, and homogenous aluminum hydroxide gel. The vaccines of the subject invention may be administered as an oil-in-water emulsion. Methods of combining adjuvants with antigens are well known to those skilled in the art.

Adjuvants may also be in particulate form. The antigen may be incorporated into biodegradable particles composed of poly-lactide-co-glycolide (PLG) or similar polymeric material. Such biodegradable particles are known to provide sustained release of the immunogen and thereby stimulate long-lasting immune responses to the immunogen. Other particulate adjuvants, include but are not limited to, micellular mixtures of Quil A and cholesterol known as immunostimulating complexes (ISCOMs) and aluminum or iron oxide beads. Methods for combining antigens and particulate adjuvants are well known to those skilled in the art. It is also known to those skilled in the art that cytotoxic T lymphocyte and other cellular immune responses are elicited when protein-based immunogens are formulated and administered with appropriate adjuvants, such as ISCOMs and micron-sized polymeric or metal oxide particles.

Suitable adjuvants for nucleic acid based vaccines include, but are not limited to, Quil A, interleukin-12 delivered in purified protein or nucleic acid form, short bacterial immunostimulatory nucleotide sequence such as CpG-containing motifs, interleukin-2/Ig fusion proteins delivered in purified protein or nucleic acid form, oil in water micro-emulsions such as MF59, polymeric microparticles, cationic liposomes, monophosphoryl lipid A (MPL), immunomodulators such as Ubenimex, and genetically detoxified toxins such as E. coli heat labile toxin and cholera toxin from Vibrio. Such adjuvants and methods of combining adjuvants with antigens are well known to those skilled in the art.

As used herein, “adjuvants” include but are not limited to mineral salts (e.g. aluminum and calcium salts), emulsions and surfactant-based formulations (e.g. MF59, AS02, montanide ISA-51 and ISA-720, QS21), particulate delivery vehicles (e.g. microparticles, liposomes, and virosomes), lipopolysaccharides (LPS) and double stranded RNA (dsRNA). Adjuvants are well known to those skilled in the art. One skilled in the art would know how to make adjuvants for use with the subject invention. Various publications describe various adjuvants and methods of development of immunostimulatory compounds and vaccine formulations for use as adjuvants. For example, Pink, J. R. and Kiery, M. P., Vaccine 22: 2097-2102 (2004); Pashine, A. et al., Nature Medicine 11: S63-S68 (2005); and Hoebe, K. et al., Nature Immunology 4: 1223-1229 (2003), which are hereby incorporated by reference into this application.

In one embodiment of the vaccine, the anthrose-containing saccharide is a monosaccharide. In another embodiment the anthrose-containing saccharide is a disaccharide. In another embodiment, the disaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoside. In another embodiment, the anthrose-containing saccharide is a trisaccharide. In another embodiment the trisaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranosyl-(1→2)-α-L-rhamnopyranoside. In another embodiment, the anthrose-containing saccharide is a tetrasaccharide. In a further embodiment, the tetrasaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoyl-(1→3)-α-L-rhamnopyranosyl-(1→2)-β-L-rhamnopyranoside. In another embodiment, the tetrasaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoyl-(1→3)-α-L-rhamnopyranosyl-(1→2)-α-L-rhamnopyranoside.

In one embodiment of the vaccine, the subject is a mammal. In another embodiment, the mammal is a human.

In one embodiment of the vaccine, the vaccine comprises (i) a conjugate of an anthrose-containing saccharide in an amount effective to enhance immunity against Bacillus anthracis in a subject, wherein the anthrose-containing saccharide is conjugated to a biomolecule via a linker, and (ii) a pharmaceutically acceptable carrier. As used herein, a “linker” is synonymous with a “spacer.” A “linker” or “spacer” shall include, without limitation, either a carbohydrate or polypeptide linked to the saccharide, either covalently or noncovalently, which allows a moiety (e.g. biomolecule) to be conjugated to the saccharide.

In one embodiment of the above vaccine, the vaccine further comprises a pharmaceutically acceptable adjuvant.

In one embodiment of the vaccine, the saccharide is conjugated with a polysaccharide, a polyacrylamide, a polypeptide or other biopolymer moiety. In another embodiment, the moiety is the protective antigen (PA) of Bacillus anthracis. In a further embodiment, the moiety is Bovine Serum Albumin (BSA).

As used herein, “biopolymers” include, but are not limited to, poly(L-lysine), other poly(amino acids), poly(vinylalcohols), polyvinylpyrrolidinones, poly(acrylic acid) derivatives, polyurethanes, and polyphosphosphazenes.

This invention provides a method for vaccinating a subject against Bacillus anthracis infection comprising administering to the subject a vaccine comprising (i) an anthrose-containing saccharide in an amount effective to enhance immunity against Bacillus anthracis in the subject and (ii) a pharmaceutically acceptable carrier, in an amount effective to stimulate production of antibodies to Bacillus anthracis spores in the subject, thereby vaccinating the subject against Bacillus anthracis.

In one embodiment of the above methods, the subject is a mammal. In the preferred embodiment, the mammal is a human.

In one embodiment of the above methods, the subject is at risk of being exposed to Bacillus anthracis spores. In another embodiment of the above methods the subject has been exposed to Bacillus anthracis spores. In another embodiment, the subject is infected with Bacillus anthracis. As used herein, a subject is exposed to Bacillus anthracis spores if the subject has come into contact with such spores via any part of the subject's body (e.g., skin, lungs or blood).

This invention provides an isolated antibody which specifically binds to an epitope on an anthrose-containing saccharide. In one embodiment, the antibody is an anti-anti-idiotypic antibody.

In one embodiment of the above isolated antibody, the saccharide is a monosaccharide. In another embodiment, the saccharide is a disaccharide. In another embodiment the saccharide is a trisaccharide or tetrasaccharide. In another embodiment, the disaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoside.

In another embodiment, the trisaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranosyl-(1→2)-α-L-rhamnopyranoside.

In a further embodiment, the tetrasaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoyl-(1→3)-α-L-rhamnopyranosyl-(1→2)-β-L-rhamnopyranoside. In another embodiment, the tetrasaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoyl-(1→3)-α-L-rhamnopyranosyl-(1→2)-α-L-rhamnopyranoside.

In one embodiment, the antibody is a monoclonal antibody. In another embodiment, the antibody is a polyclonal antibody. In another embodiment, the antibody is a chimeric antibody. In another embodiment, the antibody is humanized. In a further embodiment, the antibody is a human antibody.

This invention further provides the above antibody wherein the anthrose-containing saccharide is a trisaccharide.

In another embodiment the trisaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranosyl-(1→2)-α-L-rhamnopyranoside.

In a further embodiment of isolated antibody of claim 16, anthrose-containing saccharide is a tetrasaccharide.

The tetrasaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoyl-(1→3)-α-L-rhamnopyranosyl-(1→2)-β-L-rhamnopyranoside.

The tetrasaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoyl-(1→3)-α-L-rhamnopyranosyl-(1→2)-α-L-rhamnopyranoside.

The antibody is a monoclonal antibody, a polyclonal antibody, a chimeric antibody, a humanized antibody or a human antibody

In one embodiment, the antibody is labeled with a detectable marker. In another embodiment, the detectable marker is a radioactive label, a calorimetric marker, a luminescent marker, or a fluorescent marker.

In one embodiment of the above antibody, the antibody is labeled with an antibiotic against B. anthracis.

This invention provides an isolated anti-idiotypic antibody which specifically binds to an antibody which specifically binds to an anthrose-containing saccharide.

In one embodiment of the above antibody, the anti-idiotypic antibody is a monoclonal antibody. In another embodiment, the anti-idiotypic antibody is a polyclonal antibody. In another embodiment, the anti-idiotypic antibody is a chimeric antibody. In another embodiment, the anti-idiotypic antibody is humanized. In a further embodiment, the anti-idiotypic antibody is a human antibody.

In one embodiment of the above antibody, the anti-idiotypic antibody is labeled with a detectable marker. In another embodiment, the detectable marker is a radioactive label or a calorimetric marker, a luminescent marker, or a fluorescent marker.

Polyclonal antibodies may be produced by injecting a host animal such as rabbit, rat, goat, mouse or other animal with the immunogen of this invention, e.g. a fullerene-protein conjugate, wherein the protein may be but is not limited to thyroglobulin, RSA, or BSA. The sera are extracted from the host animal and are screened to obtain polyclonal antibodies which are specific to the immunogen. Methods of screening for polyclonal antibodies are well known to those of ordinary skill in the art such as those disclosed in Harlow & Lane, Antibodies: a Laboratory Manual, (Cold Spring Harbor Laboratories, Cold Spring Harbor, N.Y.: 1988) the contents of which are hereby incorporated by reference.

The monoclonal antibodies may be produced by immunizing for example, mice with an immunogen. The mice are inoculated intraperitoneally with an immunogenic amount of the above-described immunogen and then boosted with similar amounts of the immunogen. Spleens are collected from the immunized mice a few days after the final boost and a cell suspension is prepared from the spleens for use in the fusion.

Hybridomas may be prepared from the splenocytes and a murine tumor partner using the general somatic cell hybridization technique of Kohler, B. and Milstein, C., Nature (1975) 256: 495-497. Available murine myeloma lines, such as those from the American Type Culture Collection (ATCC) P.O. Box 1549, Manassas, Va. 20108, USA, may be used in the hybridization. Basically, the technique involves fusing the tumor cells and splenocytes using a fusogen such as polyethylene glycol. After the fusion the cells are separated from the fusion medium and grown in a selective growth medium, such as HAT medium, to eliminate unhybridized parent cells. The hybridomas may be expanded, if desired, and supernatants may be assayed by conventional immunoassay procedures, for example radioimmunoassay, using the immunizing agent as antigen. Positive clones may be characterized further to determine whether they meet the criteria of the invention antibodies.

Hybridomas that produce such antibodies may be grown in vitro or in vivo using known procedures. The monoclonal antibodies may be isolated from the culture media or body fluids, as the case may be, by conventional immuno-globulin purification procedures such as ammonium sulfate precipitation, gel electrophoresis, dialysis, chromatography, and ultrafiltration, if desired.

In the practice of the subject invention any of the above-described antibodies may be labeled with a detectable marker. “Detectable markers” which function as detectable labels are well known to those of ordinary skill in the art and include, but are not limited to, a fluorescent label, a radioactive atom, a paramagnetic ion, biotin, a chemiluminescent label or a label which may be detected through a secondary enzymatic or binding step. The secondary enzymatic or binding step may comprise the use of digoxigenin, alkaline phosphatase, horseradish peroxidase, β-galactosidase, fluorescein or steptavidin/biotin. Methods of labeling antibodies are well known in the art.

This invention provides a composition comprising (i) an antibody which specifically binds to an anthrose-containing saccharide in an amount effective to enhance immunity against Bacillus anthracis in a subject and (ii) a pharmaceutically acceptable carrier.

This invention provides a method for treating a subject infected with Bacillus anthracis, which comprises administering to the subject a therapeutically effective amount of a composition comprising (i) an antibody which specifically binds to an anthrose-containing saccharide, and (ii) a pharmaceutically acceptable carrier.

This invention provides a method for reducing the likelihood of a subject being infected with Bacillus anthracis, which comprises administering to the subject a prophylactically effective amount of a composition comprising (i) an antibody which specifically binds to an anthrose-containing saccharide, and (ii) a pharmaceutically acceptable carrier, thereby reducing the likelihood of a subject being infected with Bacillus anthracis.

In one embodiment of the above method, the subject has been exposed to Bacillus anthracis spores.

This invention provides a vaccine comprising (i) an isolated anti-idiotypic antibody which specifically binds to an antibody which specifically binds to an anthrose-containing saccharide in an amount effective to enhance immunity in a subject against Bacillus anthracis spores, and (ii) a pharmaceutically acceptable adjuvant.

This invention provides a method for vaccinating a subject against Bacillus anthracis infection comprising administering to the subject an effective amount of a vaccine comprising (i) an isolated anti-idiotypic antibody which specifically binds to an antibody which specifically binds to an anthrose-containing saccharide in an amount effective to enhance immunity in a subject against Bacillus anthracis spores, and (ii) a pharmaceutically acceptable adjuvant.

In one embodiment of the above method, the subject is wherein the subject is a mammal. In another embodiment, the mammal is human.

This invention provides a method for determining whether Bacillus anthracis spores are present in a serum sample of a subject which comprises (a) contacting the serum sample with an isolated antibody which specifically binds to an epitope on an anthrose-containing saccharide under conditions that permit the formation of a complex between the isolated antibody and an anthrose-containing saccharide; and (b) Detecting the presence of the complex formed in step (a); wherein the presence of such complex indicates that Bacillus anthracis spores are present in the sample.

In one embodiment of the above method the isolated antibody is immobilized on a solid support. In another embodiment the isolated antibody is labeled with a detectable marker.

In another embodiment of the above method, the suitable sample is blood, serum, plasma, cerebrospinal fluid, lung fluid, saliva, mucous, skin and sweat.

This invention provides a method for determining whether a subject has been exposed to Bacillus anthracis which comprises (a) contacting a serum sample from the subject with an isolated antibody which specifically binds to an epitope on an anthrose-containing saccharide under conditions that permit the formation of a complex between the antibody and an anthrose-containing saccharide; and (b) detecting the presence of the complex formed in step (a), wherein the presence of such complex indicates that the subject has been exposed to Bacillus anthracis.

In one embodiment of the above method the isolated antibody is immobilized on a solid support. In another embodiment the isolated antibody is labeled with a detectable marker. In a further embodiment the subject is infected with Bacillus anthracis.

This invention provides a method for determining whether a subject is infected with Bacillus anthracis which comprises (a) contacting a serum sample from the subject with a anti-idiotypic antibody which specifically binds to an antibody which specifically binds to an anthrose-containing saccharide under conditions which permit the formation of a complex between the anti-idiotypic antibody and an antibody which specifically binds to an anthrose-containing saccharide; and (b) determining the amount of complex formed in step (a); and (c) comparing the amount of complex determined in step (b) with an amount of complex correlative with that found for a subject infected with Bacillus anthracis, thereby determining whether the subject is infected with Bacillus anthracis.

In one embodiment of the above method the anti-idiotypic antibody is immobilized on a solid support. In another embodiment the anti-idiotypic antibody is labeled with a detectable marker.

This invention provides a kit for detecting the presence of Bacillus anthracis spores in a sample comprising, in separate compartments, an isolated antibody which specifically binds to an epitope of an anthrose-containing saccharide and reagents for detecting binding of the antibody to an anthrose-containing saccharide.

In one embodiment of the above kit, the antibody is bound to a solid support.

This invention provides a kit for detecting in a sample the presence of antibodies which bind to Bacillus anthracis spores comprising, in separate compartments, an isolated anti-idiotypic antibody which specifically binds to an antibody which specifically binds to an anthrose-containing saccharide and reagents for detecting binding of the anti-idiotypic antibody to the antibody to which it specifically binds.

In one embodiment of the above kit the anti-idiotypic antibody is bound to a solid support.

This invention provides for a kit for detecting in a sample the presence of antibodies which bind to Bacillus anthracis spores comprising, in separate compartments, the isolated anti-idiotypic antibody which specifically binds to an isolated antibody which specifically binds to an anthrose-containing saccharide, wherein the anti-idiotypic antibody is labeled with a detectable marker and reagents for detecting binding of the anti-idiotypic antibody to the antibody to which it specifically binds.

In one embodiment of the above kit, The kit of claim 62, wherein the anti-idiotypic antibody is bound to a solid support.

A composition of matter comprising (i) an antibody which specifically binds to an epitope on an anthrose-containing saccharide and (ii) a detectable marker, wherein the detectable marker is conjugated to the antibody.

The above composition wherein the detectable marker is a radioactive label, a calorimetric marker, a luminescent marker, or a fluorescent marker.

A composition of matter comprising (i) an antibody which specifically binds to an epitope on an anthrose-containing saccharide and (ii) an antibiotic against Bacillus anthracis, wherein the antibiotic against Bacillus anthracis is conjugated to the antibody.

An isolated anti-idiotypic antibody which specifically binds to an antibody which specifically binds to an anthrose-containing saccharide.

The isolated anti-idiotypic antibody is a monoclonal antibody, a polyclonal antibody, a chimeric antibody, a humanized antibody, or a human antibody.

A composition of matter comprising (i) an anti-idiotypic antibody which specifically binds to an antibody which specifically binds to an anthrose-containing saccharide, and (ii) a detectable marker, wherein the detectable marker is conjugated to the antibody.

The above composition wherein the detectable marker is a radioactive label, a calorimetric marker, a luminescent marker, or a fluorescent marker.

A composition comprising (i) an antibody which specifically binds to an anthrose-containing saccharide in an amount effective to enhance immunity against Bacillus anthracis in a subject and (ii) a pharmaceutically acceptable carrier.

A method for reducing the likelihood of a subject being infected with Bacillus anthracis, which comprises administering to the subject a prophylactically effective amount of a composition comprising (i) an antibody which specifically binds to an anthrose-containing saccharide, and (ii) a pharmaceutically acceptable carrier, thereby reducing the likelihood of a subject being infected with Bacillus anthracis.

A vaccine comprising (i) an anti-idiotypic antibody which specifically binds to an antibody which specifically binds to an anthrose-containing saccharide in an amount effective to enhance immunity in a subject against Bacillus anthracis spores, and (ii) a pharmaceutically acceptable carrier.

The vaccine of above further comprising (iii) a pharmaceutically acceptable adjuvant.

A method for vaccinating a subject against Bacillus anthracis infection comprising administering to the subject an effective amount of a vaccine comprising (i) an anti-idiotypic antibody which specifically binds to an antibody which specifically binds to an anthrose-containing saccharide in an amount effective to enhance immunity in a subject against Bacillus anthracis spores, and (ii) a pharmaceutically acceptable adjuvant.

A method for determining whether Bacillus anthracis spores are present in a serum sample of a subject which comprises: (a) contacting the serum sample with an antibody which specifically binds to an epitope on an anthrose-containing saccharide under conditions that permit the formation of a complex between the antibody and an anthrose-containing saccharide; and (b) detecting the presence of the complex formed in step (a); wherein the presence of such complex indicates that Bacillus anthracis spores are present in the serum sample.

A method for determining whether a subject has been exposed to Bacillus anthracis which comprises (a) contacting a serum sample from the subject with an antibody which specifically binds to an epitope on an anthrose-containing saccharide under conditions that permit the formation of a complex between the antibody and an anthrose-containing saccharide; and (b) detecting the presence of the complex formed in step (a), wherein the presence of such complex indicates that the subject has been exposed to Bacillus anthracis.

A method for determining whether a subject is infected with Bacillus anthracis which comprises (a) contacting a serum sample from the subject with a anti-idiotypic antibody which specifically binds to an antibody which specifically binds to an anthrose-containing saccharide under conditions which permit the formation of a complex between the anti-idiotypic antibody and an antibody which specifically binds to an anthrose-containing saccharide; (b) determining the amount of complex formed in step (a); and (c) comparing the amount of complex determined in step (b) with an amount of complex correlative with that found for a subject infected with Bacillus anthracis, thereby determining whether the subject is infected with Bacillus anthracis.

A kit for detecting the presence of Bacillus anthracis spores in a sample comprising, in separate compartments, (i) an antibody which specifically binds to an epitope of an anthrose-containing saccharide and (ii) reagents for detecting binding of the antibody to an anthrose-containing saccharide.

A kit for detecting in a sample the presence of antibodies which bind to Bacillus anthracis spores comprising, in separate compartments, (i) an anti-idiotypic antibody which specifically binds to an antibody which specifically binds to an anthrose-containing saccharide, and (ii) reagents for detecting binding of the anti-idiotypic antibody to the antibody to which it specifically binds.

A kit for detecting in a sample the presence of antibodies which bind to Bacillus anthracis spores comprising, in separate compartments, (i) a conjugate of an anti-idiotypic antibody which specifically binds to an antibody which specifically binds to an anthrose-containing saccharide, wherein the anti-idiotypic antibody is conjugated to a detectable marker and (ii) reagents for detecting binding of the anti-idiotypic antibody to the antibody to which it specifically binds.

This invention will be better understood from the Experimental Details that follow. However, one skilled in the art will readily appreciate that the specific methods and results discussed are merely illustrative of the invention as described more fully in the claims which follow thereafter.

First Series of Experiments

Synopsis

Using photo-generated glycan arrays, we characterized a large-panel of synthetic carbohydrates for their antigenic reactivities with pathogen-specific antibodies. We discovered that rabbit IgG antibodies elicited by B. anthracis spores specifically recognize a tetrasaccharide chain that decorates the outermost surfaces of the B. anthracis exosporium. Since this sugar moiety is highly specific for the spores of B. anthracis, it appears to be a key biomarker for detection of B. anthracis spores and development of novel vaccines that target anthrax spores.

Experimental Approach

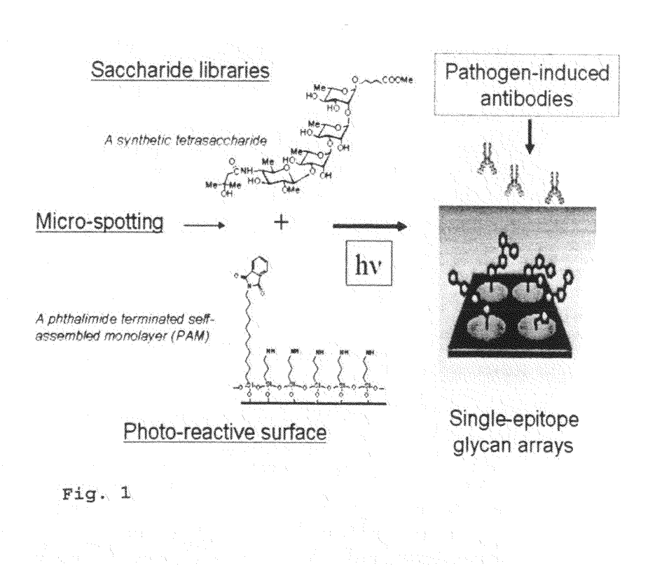

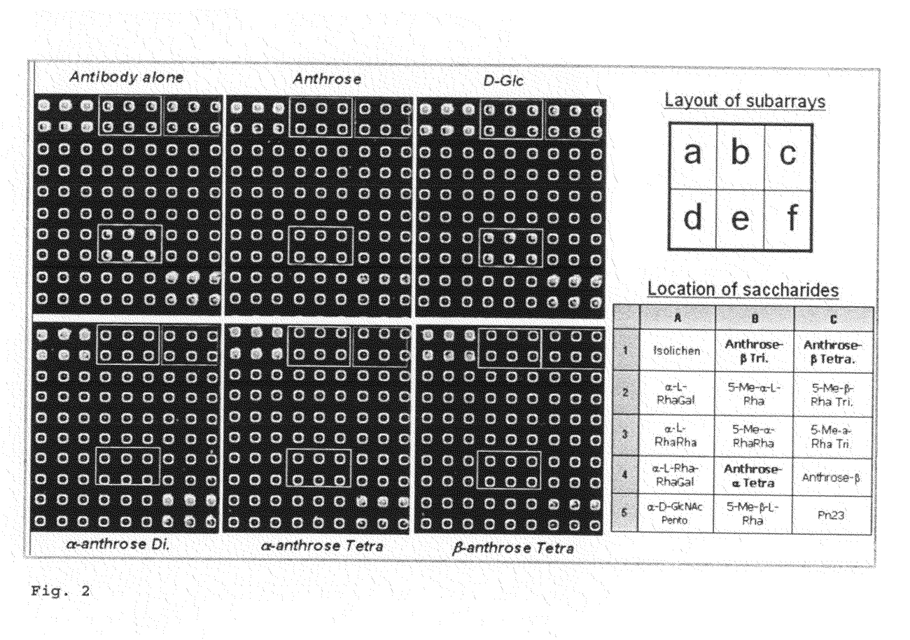

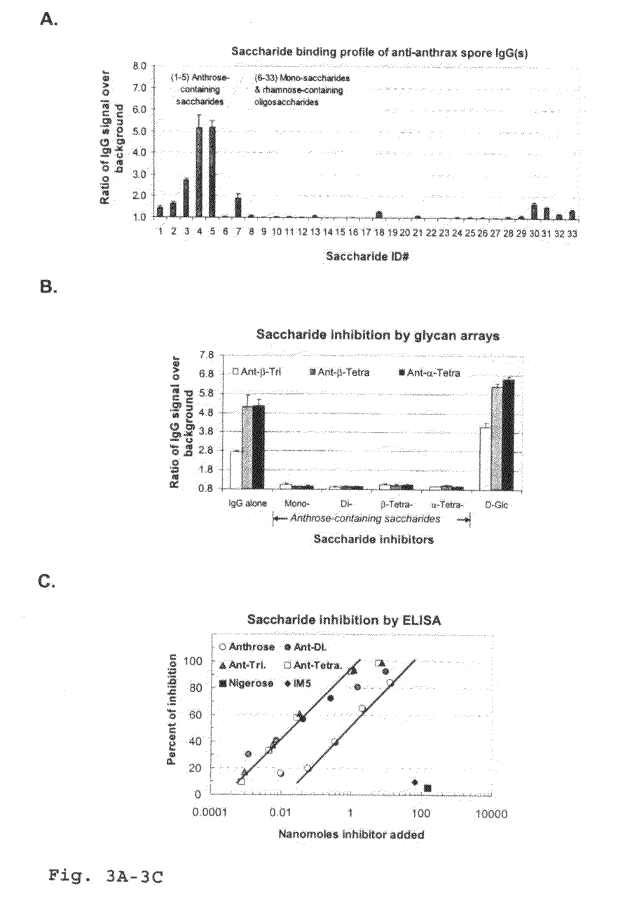

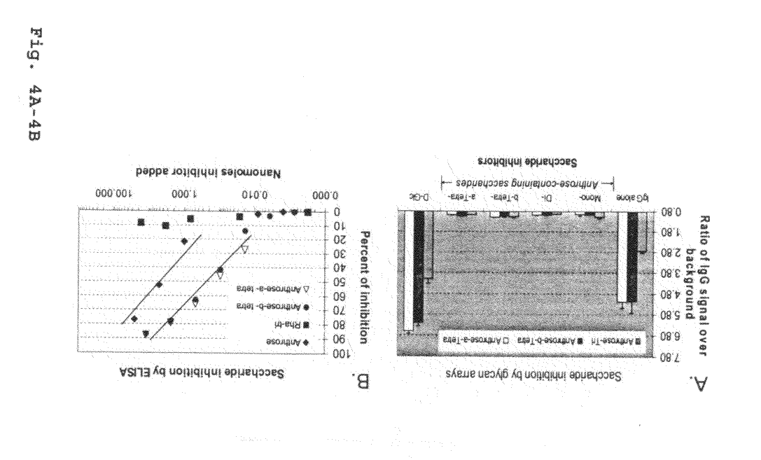

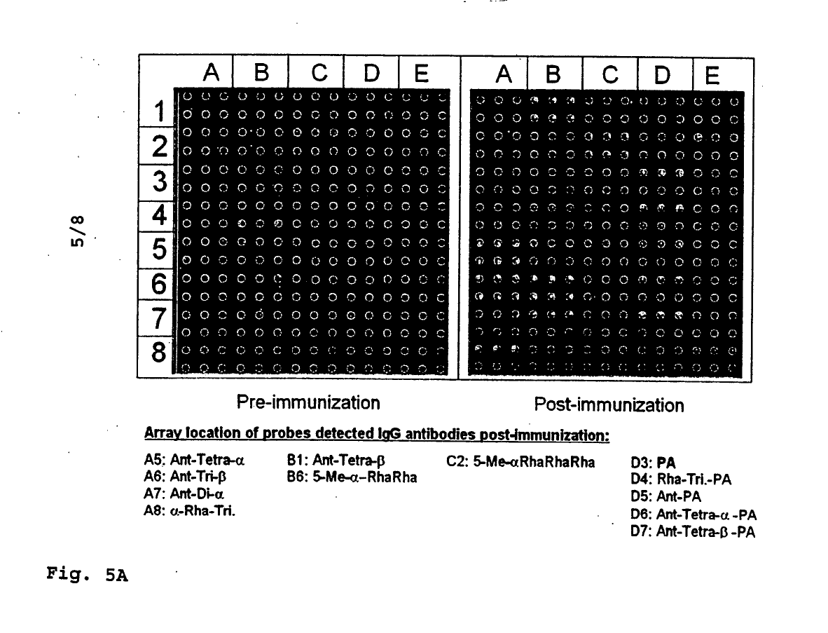

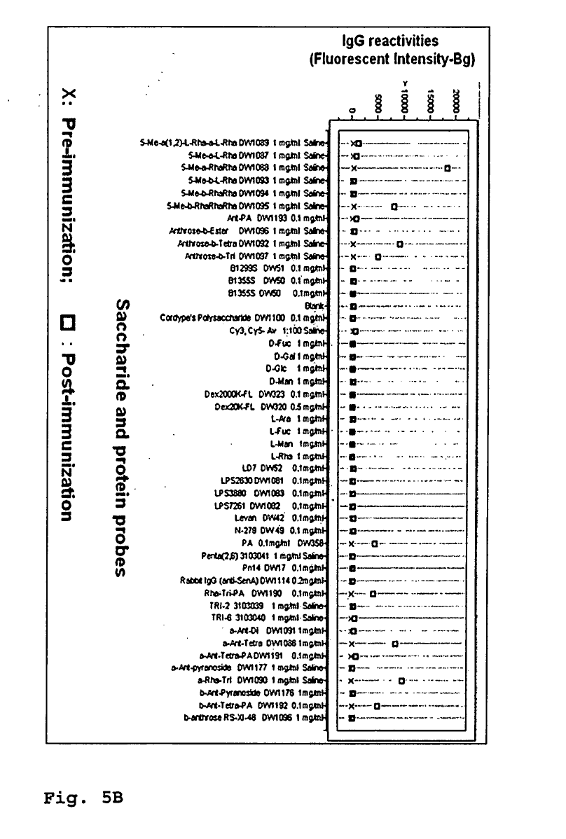

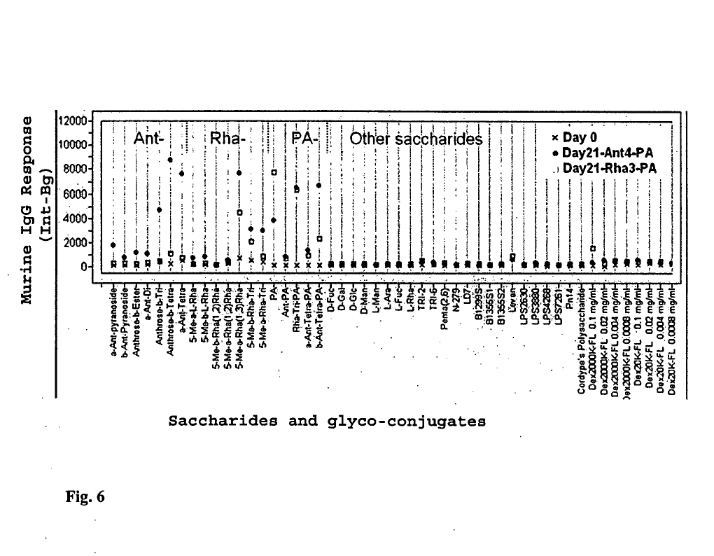

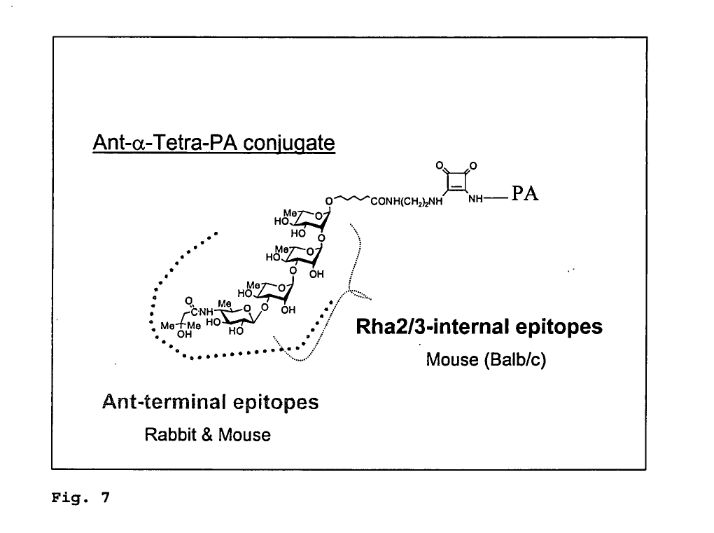

We present here a high-throughput strategy to facilitate identification and immunological characterization of pathogen-specific carbohydrate moieties, including those of the B. anthracis exosporium. This strategy employs highly sensitive oligosaccharide microarrays to probe specific antibodies that were elicited by infections or immunizations. Our rationale was that if a pathogen expressed immunogenic carbohydrate structures, then immunizing animals using this microbe or its antigen preparations would have the possibility to elicit antibodies specific for these structures. Characterizing these antibody responses using a broad-range glycan array that displays the corresponding saccharides may thus rapidly identify the immunogenic sugar moieties of the corresponding pathogens. A schematic overview of this biomarker identification strategy is shown in FIG. 1.

I. Materials and Methods

Microarray Construction

Antigen preparations were dissolved in saline (0.9% NaCl) at a given concentration and were spotted as triplet replicate spots in parallel. The initial amount of antigen spotted was approximately 0.35 ng per spot and diluted by 1:5 in saline. A high-precision robot designed to produce cDNA microarray (PIXSYS 5500C, Cartesian Technologies Irvine, Calif.) was utilized to spot carbohydrate antigens onto chemically modified glass slides as described [15, 18, 22, 23]. Both FAST Slides (Schleicher & Schuell, Keene, N.H.) and the phthalimide-amine monolayers (PAM)-coated slides were spotted. The printed FAST slides were air-dried and stored at room temperature. The printed PAM slides were subjected to UV irradiation in order to activate the photo-coupling of carbohydrates to the surface.

Photo-Coupling of Carbohydrates on the Chips

After microarray spotting, the PAM slides were air-dried and placed in a quartz tube. The sealed tube was subsequently purged with argon or nitrogen before irradiation. UV irradiation was conducted by placing the quartz tube under a desktop lamp containing a 300 nm Rayonet bulb for one hour. Precaution was made to avoid skin and eye contact with the radiation during the irradiation process.

Microarray Binding and Inhibition Assays

Immediately before use, the printed microarrays were rinsed twice with 1×PBS (PH 7.4). They were then “blocked” by incubating the slides in 1% BSA in 1×PBS with 0.025% NaN3 and 0.05% Tween 20 (PBST) at room temperature (RT) for 30 minutes. Rabbit anti-B. anthracis IgG antibodies rabbit (Catalog No. B0003-05G, United States Biological, Swampscott, Mass. and Catalog No. ab8244, Abcam Limited, Cambridge, UK) were applied at proper dilutions in 1% BSA PBST as specified in the figure legend. Staining was conducted at RT with one hour incubation in a humidity chamber. For inhibition assay, antibodies were pre-incubated with a saccharide inhibitor at 37° C. for 1 hour before glycan array staining. A biotinylated anti-rabbit IgG antibody (Sigma) was applied to reveal the bound IgG and subsequently stained with streptavidin-Cy5 conjugate (Amersham Pharmacia). The stained slides were rinsed five times with PBST after each staining step. A ScanArray 5000A microarray scanner (PerkinElmer, Torrance, Calif.) equipped with multiple lasers, emission filters and ScanArray Acquisition Software was used to scan the microarray. Fluorescence intensity values for each array spot and its background were calculated and statistically analyzed using ScanArray Express Software package (PerkinElmer, Torrance, Calif.).

2. Results

Photo-generation of epitope-specific glycan arrays is a key component of this strategy. We utilized a photo-active surface for covalent immobilization and micro-patterning of carbohydrates onto glass substrates[15]. This method employs a glass slide coated with a self-assembled mixed monolayer that presents photo-active phthalimide chromophores at the air-monolayer interface[15]. Upon exposure to UV radiation (300 nm), the phthalimide end-groups graft the spotted carbohydrates by hydrogen abstraction followed by radical recombination. The efficacy of carbohydrate immobilization is independent of the molecular weights of spotted carbohydrates[15]. Thus, a unique technical advantage of this method is the ability to produce epitope-specific glycan arrays using unmodified mono- and oligo-saccharides. We applied, therefore, this technology to display a large panel of saccharide structures, including synthetic fragments and derivatives of the anthrose-containing tetrasaccharide side chain of the B. anthracis exosporium [12, 16-18] and a number of control carbohydrate antigens (Supplementary Table 1), for an immunological characterization.

At the onset of this work we assumed that if B. anthracis spores express potent immunogenic carbohydrate moieties, immunization with the spores would elicit antibodies specific for these sugar structures. Such antibody reactivities would then be detected by glycan arrays that display the corresponding sugar structures. Therefore, we incubated the anthrax saccharide glycan arrays with rabbit anti-B. anthracis spore antibodies and examined the potential presence of anti-carbohydrate specificities.

FIG. 2 shows a glycan array image that was stained with a preparation of pooled rabbit polyclonal anti-anthrax spore IgG antibodies. As highlighted with colored boxes in the image, a number of immobilized carbohydrate probes detected significant amounts of IgG antibodies. These include an anthrose-containing trisaccharide, and both the β-tetrasaccharide and α-tetrasaccharide in the form of their methyl 6-hydroxyhexanoyl glycosides. The glycan array also shows other anti-carbohydrate antibody activities, such as anti-polysaccharide antibodies for isolichenin [α(1,3)Glucan] (positive spots in the upper-left corner) and Streptococcus pneumoniae type 23 (Pn 23) polysaccharide (positive spots in the bottom-right corner). These reagents were spotted on the chips as positive controls for the assay system since we detected a significant amount of IgG antibodies for these antigens in this preparation of rabbit polyclonal IgGs in a preliminary experiment.

FIG. 3a illustrates a quantitative comparison of the relative antibody reactivities detected by the glycan arrays. The antibody reactivity with anthrose-containing saccharides correlates positively with the sizes of the saccharides immobilized on the glycan arrays. The anthrose monosaccharide and anthrose-containing disaccharide are marginally reactive while the anthrose-containing tetrasaccharides are highly reactive, regardless of their configuration at the anomeric center carrying the aglycone. Such antibody reactivities were not detected in a collection of four pre-immunized rabbit sera and twelve rabbit anti-sera against irrelevant antigens. These results demonstrate that rabbit IgG antibodies elicited by the native spore antigens recognize the synthetic carbohydrate moieties of the BclA glycoprotein.

In order to grade the immunodominance of sugar moieties of the B. anthracis exosporium, we conducted microarray-based saccharide blocking assays (FIG. 3b). The microarray assay simultaneously measures the relative antigenic reactivities of antibodies with multiple antigenic structures. Introducing a specific saccharide as a competitor into this multiplex antigen-antibody interaction provides a critical measurement of the specificity and cross-reactivity of a specific antibody preparation. If an antibody fingerprint, i.e., an array of positively stained micro-spots of a given saccharide structure, would be blocked by a specific saccharide, we could infer that the specificity of the reacting antibody is specific for the corresponding saccharide structure. Inhibition of a number of different antibody fingerprints by a given saccharide would suggest that these reactive saccharide structures share common or cross-reactive sugar epitopes.

We scanned a panel of monosaccharides and glycosides to identify potential inhibitors, including 5-methoxycarbonyl β-anthroside, L-rhamnose, and other common sugar residues listed in the supplementary table 1. Only the anthrose glycoside blocked the antibody reactivity with anthrose-containing tri- and tetra-saccharides (α and β glycosides) (FIG. 2 and FIGS. 3A-3C). In contrast, the same anthrose glycoside shows no significant inhibition of other antibody reactivities observed with the same rabbit polyclonal antibodies, such as binding to the polysaccharide isolichenin [α(1,3)-glucan] (FIG. 2, positive spots in the upper-left corner) and a preparation of Pn 23 polysaccharide (FIG. 2, positive spots in the bottom-right corner). We also examined anthrose-containing di, tri, and tetra-saccharides and observed that their inhibition profiles or specificities are identical to those mediated by the anthrose monosaccharide, i.e., they competitively inhibit the specific antibody reactivities with the anthrose-containing saccharide moieties but not other anti-carbohydrate reactivities present in the polyclonal rabbit IgG preparation.

In order to determine the relative contribution of sugar residues of the anthrose-containing tetrasaccharide to the antibody binding, we performed ELISA-based quantitative saccharide inhibition assays using anthrose and anthrose-containing di-, tri-, and tetra-saccharides. In this assay, an anthrose-tetrasaccharide-BSA conjugate was coated on an ELISA plate and the corresponding saccharides were mixed with anti-anthrax spore IgG antibodies in the binding reaction. As shown in FIG. 3C, the quantitative inhibition curves generated by the four saccharides are nearly linear. The curve of the anthrose-trisaccharide (IC₅₀, 0.016 nmol) is essentially superimposed to the curve of the anthrose-tetrasaccharide (IC₅₀, 0.016 nmol). Both marginally differ from the disaccharide (IC₅₀, 0.019 nmols) but are significantly different from the monosaccharide (IC₅₀, 0.688 nmols). The relative inhibiting powers of the four anthrose saccharides are, thus, 1.00(Tetra)/1.00(Tri.)/1.18(Di.)/43.3(Mono.). In striking contrast, an α(1,3)glucosyl disaccharide, Nigerose, and an α(1,6)glucosyl pentasaccharide, IM5 (isomaltopentose), show no inhibition to the specific antibody reactivities with the anthrose-tetrasaccharide in the same assay.

Taking together, we demonstrate that IgG antibodies elicited by the native antigens of the B. anthracis spore recognize synthetic anthrose-containing sugar moieties. The saccharide binding reactivities correlate directly with the sizes of the saccharides displayed by the glycan arrays. The terminal anthrose monosaccharide is marginally reactive and the anthrose-containing tetrasaccharides highly reactive, regardless of their anomeric configuration. However, the smaller saccharide units, including the anthrose mono-, di-, and tri-saccharides are potent inhibitors of the specific antibody reactivities to the tetrasaccharides displayed either by the photo-generated glycan arrays or by a BSA-conjugate on an ELISA microtiter plate. We conclude, therefore, that the anthrose-containing tetrasaccharide is immunogenic in its native configuration as displayed by the exosporium BclA glycoprotein and its terminal trisaccharide unit is essential for the constitution of an highly specific antigenic determinant.

Given the fact that this carbohydrate moiety is displayed on the outermost surfaces of B. anthracis spores [11, 12] and its expression is highly specific for the spore of B. anthracis[12], the anthrose-containing tetrasaccharide can be considered an important immunological target. Its applications may include identification of the presence of B. anthracis spores, surveillance and diagnosis of anthrax infection and development of novel vaccines targeting the B. anthracis spore. Effort must also be made to explore the biological role of this highly specific carbohydrate moiety of B. anthracis. Generally, the experimental approach we demonstrated here allows high-throughput screening of libraries of saccharide structures for their potential antigenic reactivities. Its application may substantially facilitate the identification of key immunogenic sugar moieties of microbial pathogens.

SUPPLEMENTARY TABLE 1
Saccharides used in glycan array characterization (FIG. 2-4)

| ID# | Abbreviation | Structural Information | Source | Reference |
|---|---|---|---|---|
| 1 | Ant | 5-(Methoxycarbonyl)pentyl 4-(3-hydroxy-3- | Present authors | Saksena, R. et al., Carbohydr Res |
| | | methylbutanamido)-2-O-methyl-4,6-dideoxy-β-D- | | 340, 1591-1600 (2005). |
| | | glucopyranoside | | |
| 2 | Ant-α-Rha | 5-(Methoxycarbonyl)pentyl 4-(3-hydroxy-3- | Present authors | Saksena, R. et al., Manuscript in |
| | | methylbutanamido)-2-O-methyl-4,6-dideoxy-β-D- | | preparation. |
| | | glucopyranosyl-(1→3)-α-L-rhamnopyranoside | | |
| 3 | Ant-(1→3)-α-Rha- | 5-(Methoxycarbonyl)pentyl 4-(3-hydroxy-3- | Present authors | Saksena, R. et al., Manuscript in |
| | (1→2)-α-Rha | methylbutanamido)-2-O-methyl-4,6-dideoxy-β-D- | | preparation. |
| | | glucopyranosyl-(1→3)-α-L-rhamnopyranosyl-(1→2)-α-L- | | |
| | | rhamnopyranoside | | |
| 4 | Ant-(1→3)-α-Rha- | 5-(Methoxycarbonyl)pentyl 4-(3-hydroxy-3- | Present authors | Adamo, R. et al., Carbohydr Res 340, |
| | (1→3)-α-Rha-(1→2)- | methylbutanamido)-2-O-methyl-4,6-dideoxy-β-D- | | 2579-2582 (2005); |
| | β-Rha | glucopyranosyl-(1→3)-α-L-rhamnopyranoyl-(1→3)-α-L- | | |
| | | rhamnopyranosyl-(1→2)-β-L-rhamnopyranoside | | |
| 5 | Ant-(1→3)-α-Rha- | 5-(Methoxycarbonyl)pentyl 4-(3-hydroxy-3- | Present authors | Saksena, R. et al., Bioorg Med Chem |
| | (1→3)-α-Rha-(1→2)- | methylbutanamido)-2-O-methyl-4,6-dideoxy-β-D- | | Lett 16, 615-617 (2006). |
| | α-Rha | glucopyranosyl-(1→3)-α-L-rhamnopyranoyl-(1→3)-α-L- | | |
| | | rhamnopyranosyl-(1→2)-α-L-rhamnopyranoside | | |
| 6 | Rha | L-Rhamnose | Sigma | |
| 7 | Fuc | D-Fucose | Sigma | |
| 8 | Gal | D-Galactose | Sigma | |
| 9 | Glc | D-Glucose | Sigma | |
| 10 | Man | D-Mannose | Sigma | |
| 11 | GalNAc | N-acetyl-2-amino-2-deoxy-D-galactose | Sigma | |
| 12 | Ara | L-Arabinose | Sigma | |
| 13 | Man | L-Mannose | Sigma | |
| 14 | α-Rha | 5-(Methoxycarbonyl)pentyl α-L-rhamnopyranoside | Present authors | Saksena, R. et al., Manuscript in |
| | | | | preparation |
| 15 | β-Rha | 5-(Methoxycarbonyl)pentyl β-L-rhamnopyranoside | Present authors | Adamo, R. et al., Helv. Chim. Acta, |
| | | | | accepted for publication. |
| 16 | α-Rha-(1→3)-α-Rha | 5-(Methoxycarbonyl)pentyl α-L-rhamnopyranosyl-(1→3)- | Present authors | Saksena, R. et al., Manuscript in |
| | | α-L-rhamnopyranoside | | preparation. |
| 17 | α-Rha-(1→3)-α-Rha- | 5-(Methoxycarbonyl)pentyl α-L-rhamnopyranosyl-(1→3)- | Present authors | Saksena, R. et al., Manuscript in |
| | (1→2)-α-Rha | α-L-rhamnopyranosyl-(1→2)-α-L-rhamnopyranoside | | preparation. |
| 18 | Rha-(1→2)-α-Rha | 5-(Methoxycarbonyl)pentyl α-L-rhamnopyranosyl-(1→2)- | Present authors | Saksena, R. et al., Manuscript in |
| | | α-L-rhamnopyranoside | | preparation. |
| 19 | Rha-(1→2)-β-Rha | 5-(Methoxycarbonyl)pentyl α-L-rhamnopyranosyl-(1→2)- | Present authors | Adamo, R. et al., Helv. Chim. Acta, |
| | | β-L-rhamnopyranoside | | accepted for publication. |
| 20 | α-Rha-(1→3)-α-Rha- | 5-(Methoxycarbonyl)pentyl α-L-rhamnopyranosyl-(1→3)- | Present authors | Adamo, R. et al., Helv. Chim. Acta, |
| | (1→2)-β-Rha | α-L-rhamnopyranosyl-(1→2)-β-L-rhamnopyranoside | | accepted for publication. |
| 21 | α-Rha-(1→2)-Gal | Methyl <-L-rhamnopyranosyl-(1→2)-<-D-galactopyranoside | Present authors | Ková\check{c} et. al. J. Org. Chem., 57, |
| | | | | 2455-2467 (1992). |
| 22 | Me α-GlcNAc-(1→3)-α- | Methyl 2-acetamido-2-deoxy-<-D-glucopyranosyl-(1→3)-<- | Present authors | Pavliak et. al., Carbohydr. Res., 229, |
| | Rha-(1→3)-α-Rha- | L-rhamnopyranosyl-(1→3)-<-L-rhamnopyranosyl-(1→2)-<- | | 103-116 (1992). |
| | (1→2)-α-Gal | D-galactopyranoside | | |
| 23 | Me α-Rha-(1→2)-α-Gal- | Methyl <-L-rhamnopyranosyl-(1→2)-<-D- | Present authors | Ková\check{c} & Edgar J. Org. Chem., 57, |
| | (1→3)-α-GlcNAc | galactopyranosyl-(1→3 )-2-acetamido-2-deoxy-<-D- | | 2455-2467 (1992). |
| | | glucopyranoside | | |
| 24 | Me α-Rha-(1→3)-α-Rha | Methyl <-L-rhamnopyranosyl-(1→3)-<-L- | Present authors | Ková\check{c} & Edgar J. Org. Chem., 57, |
| | | rhamnopyranoside | | 2455-2467 (1992) |
| 25 | Me α-Rha-(1→3)-α- | Methyl <-L-rhamnopyranosyl-(1→3)-<-L-rhamnopyranosyl- | Present authors | Ková\check{c} & Edgar J. Org. Chem., 57, |
| | Rha-(1→2)-α-Gal | (1→2)-<-D-galactopyranoside | | 2455-2467 (1992) |
| 26 | Me α-Rha-(1→3)-α- | Methyl <-L-rhamnopyranosyl-(1→3)-<-L-rhamnopyranosyl- | Present authors | Ková\check{c} & Edgar J. Org. Chem., 57, |
| | Rha-(1→2)-α-Gal- | (1→2)-<-D-galactopyranosyl-(1→3)-2-acetamido-2-deoxy- | | 2455-2467 (1992) |
| | (1→3)-α-GlcNAc | <-D-glucopyranoside | | |
| 27 | Ogawa-Tri | Methyl 2-O-methyl-4,6-dideoxy-4-(3-deoxy-L-glycero- | Present authors | Zhang, J. & Kovac, P., Carbohydr. |
| | | tetronamido)-<-D-mannopyranosyl-(1→2)-4,6-dideoxy-4- | | Res 300, 329-339 (1997) |
| | | (3-deoxy-L-glycero-tetronamido)-<-D-mannopyranosyl- | | |
| | | (1→2)-4,6-dideoxy-4-(3-deoxy-L-glycero-tetronamido)-<- | | |
| | | D-mannopyranoside | | |
| 28 | Ogawa-Tetra | Methyl 2-O-methyl-4,6-dideoxy-4-(3-deoxy-L-glycero- | Present authors | Zhang, J. & Kovac, P., Carbohydr. |
| | | tetronamido)-<-D-mannopyranosyl-(1→2)-bis-[4,6-dideoxy- | | Res 300, 329-339 (1997) |
| | | 4-(3-deoxy-L-glycero-tetronamido)-<-D-mannopyranosyl- | | |
| | | (1→2)]-4,6-dideoxy-4-(3-deoxy-L-glycero-tetronamido)-<- | | |
| | | D-mannopyranoside | | |
| 29 | Ogawa-Penta | Methyl 2-O-methyl-4,6-dideoxy-4-(3-deoxy-L-glycero- | Present authors | Zhang, J. & Kovac, P., Carbohydr. |
| | | tetronamido)-<-D-mannopyranosyl-(1→2)-tris[4,6-dideoxy- | | Res 300, 329-339 (1997) |
| | | 4-(3-deoxy-L-glycero-tetronamido)-<-D-mannopyranosyl- | | |
| | | (1→2)]-4,6-dideoxy-4-(3-deoxy-L-glycero-tetronamido)-<- | | |
| | | D-mannopyranoside | | |
| 30 | Ogawa-Hexa | Methyl 2-O-methyl-4,6-dideoxy-4-(3-deoxy-L-glycero- | Present authors | Zhang, J. & Kovac, P., Carbohydr. |
| | | tetronamido)-<-D-mannopyranosyl-(1→2)-tetrakis[4,6- | | Res 300, 329-339 (1997) |
| | | dideoxy-4-(3-deoxy-L-glycero-tetronamido)-<-D- | | |
| | | mannopyranosyl-(1→2)]-4,6-dideoxy-4-(3-deoxy-L- | | |
| | | glycero-tetronamido)-<-D-mannopyranoside | | |
| 31 | Ogawa-Tri-BSA | [Methyl 2-O-methyl-4,6-dideoxy-4-(3-deoxy-L-glycero- | Present authors | Saksena et. al., Carbohydr. Res., 338, |
| | | tetronamido)-<-D-mannopyranosyl-(1→2)-4,6-dideoxy-4- | | 2591-2603 (2003). |
| | | (3-deoxy-L-glycero-tetronamido)-<-D-mannopyranosyl- | | |
| | | (1→2)-4,6-dideoxy-4-(3-deoxy-L-glycero-tetronamido)-<- | | |
| | | D-mannopyranoside]₅-BSA | | |
| 32 | Ogawa-Tetra-BSA | [Methyl 2-O-methyl-4,6-dideoxy-4-(3-deoxy-L-glycero- | Present authors | Saksena et. al., Carbohydr. Res., 338, |
| | | tetronamido)-<-D-mannopyranosyl-(1→2)-bis[4,6-dideoxy- | | 2591-2603 (2003). |
| | | 4-(3-deoxy-L-glycero-tetronamido)-<-D-mannopyranosyl- | | |
| | | (1→2)]-4,6-dideoxy-4-(3-deoxy-L-glycero-tetronamido)-<- | | |
| | | D-mannopyranoside]₁₀-BSA | | |
| 33 | Ogawa-Penta-BSA | [Methyl 2-O-methyl-4,6-dideoxy-4-(3-deoxy-L-glycero- | Present authors | Saksena et. al., Carbohydr. Res., 338, |
| | | tetronamido)-<-D-mannopyranosyl-(1→2)-tris[4,6-dideoxy- | | 2591-2603 (2003). |
| | | 4-(3-deoxy-L-glycero-tetronamido)-<-D-mannopyranosyl- | | |
| | | (1→2)]-4,6-dideoxy-4-(3-deoxy-L-glycero-tetronamido)-<- | | |
| | | D-mannopyranoside]₁₀-BSA | | |
| 34 | Isolichenin | α-(1→3)-Glucan | From the | Heidelberger M., J Immunol.; |
| | | | collection of | 91:735-9, (1963) |
| | | | late Prof. Elvin | |
| | | | A. Rabat, | |
| | | | Columbia | |
| | | | University | |
| 35 | Pn23 | Streptococcus pneumoniae type 23 capsular polysaccharide | From the | Roy, A. & Roy, N., Carbohydrate |
| | | (D-Gal:D-Glu:L-Rha:Glycerol:Phdsphorus = 1:1:2:0.6:1 and | collection of | Res., 126: 271-7, (1984); |
| | | with the terminal L-Rha as a key residue of a dominant | late Prof. Elvin | Heidelberger M. et al., J Immunol.; |
| | | antigenic determinant) | A. Kabat, | 99: 794-6, (1967). |
| | | | Columbia | |
| | | | University | |

SUPPLEMENTARY TABLE 2
Glycan array data of FIG. 2

| | | *IgG reactivities | | |
|---|---|---|---|---|
| | | (Ratio of Sg./bg.) | | |
| Saccharides spotted | | Array | | |
| Id* | Short name** | location | Mean | SD |
| Subarray-a, IgG 20 μg/ml: No | | | | |
| Inhibitor | | | | |
| 34 | Isolichenin | A1 | 36.32 | 1.52 |
| 21 | Me α-L-RhaGal | A2 | 1.08 | 0.05 |
| 24 | Me L-α-Rha((1→3)Rha | A3 | 1.02 | 0.04 |
| 25 | Me α-L-RhaRhaGal | A4 | 1.02 | 0.04 |
| 22 | Me α-D-GlcNAcRhaRhaGal | A5 | 0.95 | 0.03 |
| 3 | 5-Methoxycarbonyl Anthrose-α-Tri | B1 | 2.73 | 0.09 |
| 14 | 5-Methoxycarbonyl α-L-Rha | B2 | 0.97 | 0.03 |
| 16 | 5-Methoxycarbonyl αRha(1→3)Rha | B3 | 1.00 | 0.04 |
| 5 | 5-Methoxycarbonyl Anthrose-α-Tetra | B4 | 5.19 | 0.31 |
| 15 | 5-Methoxycarbonyl β-L-Rha | B5 | 0.95 | 0.01 |
| 4 | 5-Methoxycarbonyl Anthrose-β-Tetra | C1 | 5.16 | 0.58 |
| 20 | 5-Methoxycarbonyl β-RhaRhaRha | C2 | 0.95 | 0.04 |
| 17 | 5-Methoxycarbonyl α-RhaRhaRha | C3 | 0.98 | 0.01 |
| 1 | 5-Methoxycarbonyl Anthrose | C4 | 1.44 | 0.08 |
| 35 | Pn23 polysaccharide | C5 | 12.35 | 5.24 |
| Subarray-b, IgG 10 μg/ml: 5-Methoxycarbonyl Anthrose 0.25 mg/ml | | | | |
| 34 | Isolichenin | A1 | 11.85 | 0.02 |
| 21 | Me α-L-RhaGal | A2 | 0.96 | 0.06 |
| 24 | Me L-α-Rha((1→3)Rha | A3 | 0.96 | 0.04 |
| 25 | Me α-L-RhaRhaGal | A4 | 0.97 | 0.04 |
| 22 | Me α-D-GlcNAcRhaRhaGal | A5 | 0.96 | 0.02 |
| 3 | 5-Methoxycarbonyl Anthrose-α-Tri | B1 | 1.08 | 0.05 |
| 14 | 5-Methoxycarbonyl α-L-Rha | B2 | 0.93 | 0.03 |
| 16 | 5-Methoxycarbonyl αRha(1→3)Rha | B3 | 0.95 | 0.04 |
| 5 | 5-Methoxycarbonyl Anthrose-α-Tetra | B4 | 0.96 | 0.07 |
| 15 | 5-Methoxycarbonyl β-L-Rha | B5 | 0.93 | 0.07 |
| 4 | 5-Methoxycarbonyl Anthrose-β-Tetra | C1 | 0.96 | 0.02 |
| 20 | 5-Methoxycarbonyl β-RhaRhaRha | C2 | 1.04 | 0.05 |
| 17 | 5-Methoxycarbonyl α-RhaRhaRha | C3 | 0.97 | 0.05 |
| 1 | 5-Methoxycarbonyl Anthrose | C4 | 0.93 | 0.03 |
| 35 | Pn23 polysaccharide | C5 | 5.27 | 1.36 |
| Subarray-C, IgG 20 μg/ml: | D-Glu 0.25 mg/ml | | | |
| 34 | Isolichenin | A1 | 34.88 | 1.04 |
| 21 | Me α-L-RhaGal | A2 | 1.14 | 0.00 |
| 24 | Me L-α-Rha((1→3)Rha | A3 | 0.99 | 0.01 |
| 25 | Me α-L-RhaRhaGal | A4 | 1.04 | 0.06 |
| 22 | Me α-D-GlcNAcRhaRhaGal | A5 | 1.06 | 0.04 |
| 3 | 5-Methoxycarbonyl Anthrose-α-Tri | B1 | 4.09 | 0.24 |
| 14 | 5-Methoxycarbonyl α-L-Rha | B2 | 1.03 | 0.01 |
| 16 | 5-Methoxycarbonyl αRha(1→3)Rha | B3 | 1.05 | 0.05 |
| 5 | 5-Methoxycarbonyl Anthrose-α-Tetra | B4 | 6.60 | 0.13 |
| 15 | 5-Methoxycarbonyl β-L-Rha | B5 | 1.07 | 0.03 |
| 4 | 5-Methoxycarbonyl Anthrose-β-Tetra | C1 | 6.21 | 0.17 |
| 20 | 5-Methoxycarbonyl β-RhaRhaRha | C2 | 1.04 | 0.05 |
| 17 | 5-Methoxycarbonyl α-RhaRhaRha | 03 | 1.00 | 0.03 |
| 1 | 5-Methoxycarbonyl Anthrose | C4 | 1.15 | 0.01 |
| 35 | Pn23 polysaccharide | C5 | 36.14 | 4.17 |
| Subarray-d, IgG 10 μg/ml: | 5-Methoxycarbonyl Anthrose-α-DI 0.25 mg/ml | | | |
| 34 | Isolichenin | A1 | 11.72 | 3.67 |
| 21 | Me α-L-RhaGal | A2 | 0.98 | 0.03 |
| 24 | Me L-α-Rha((1→3)Rha | A3 | 0.99 | 0.06 |
| 25 | Me α-L-RhaRhaGal | A4 | 0.98 | 0.04 |
| 22 | Me α-D-GlcNAcRhaRhaGal | A5 | 0.98 | 0.01 |
| 3 | 5-Methoxycarbonyl Anthrose-α-Tri | B1 | 0.95 | 0.02 |
| 14 | 5-Methoxycarbonyl α-L-Rha | B2 | 0.94 | 0.04 |
| 16 | 5-Methoxycarbonyl αRha(1→3)Rha | B3 | 1.04 | 0.10 |
| 5 | 5-Methoxycarbonyl Anthrose-α-Tetra | B4 | 0.94 | 0.02 |
| 15 | 5-Methoxycarbonyl β-L-Rha | B5 | 0.98 | 0.03 |
| 4 | 5-Methoxycarbonyl Anthrose-β-Tetra | C1 | 0.93 | 0.04 |
| 20 | 5-Methoxycarbonyl β-RhaRhaRha | C2 | 0.94 | 0.03 |
| 17 | 5-Methoxycarbonyl α-RhaRhaRha | C3 | 0.99 | 0.01 |
| 1 | 5-Methoxycarbonyl Anthrose | C4 | 0.99 | 0.04 |
| 35 | Pn23 polysaccharide | C5 | 26.34 | 3.18 |
| Subarray-e, IgG 20 μg/ml: | 5-Methoxycarbonyl Anthrose-α-Tetra 0.25 mg/ml | | | |
| 34 | Isolichenin | A1 | 30.65 | 1.07 |
| 21 | Me α-L-RhaGal | A2 | 1.09 | 0.06 |
| 24 | Me L-α-Rha((1→3)Rha | A3 | 0.98 | 0.08 |
| 25 | Me α-L-RhaRhaGal | A4 | 1.01 | 0.02 |
| 22 | Me α-D-GlcNAcRhaRhaGal | A5 | 1.01 | 0.04 |
| 3 | 5-Methoxycarbonyl Anthrose-α-Tri | B1 | 0.96 | 0.02 |
| 14 | 5-Methoxycarbonyl α-L-Rha | B2 | 0.99 | 0.04 |
| 16 | 5-Methoxycarbonyl αRha(1→3)Rha | B3 | 0.95 | 0.04 |
| 5 | 5-Methoxycarbonyl Anthrose-α-Tetra | B4 | 0.98 | 0.02 |
| 15 | 5-Methoxycarbonyl β-L-Rha | B5 | 1.00 | 0.03 |
| 4 | 5-Methoxycarbonyl Anthrose-β-Tetra | C1 | 1.01 | 0.03 |
| 20 | 5-Methoxycarbonyl β-RhaRhaRha | C2 | 0.99 | 0.06 |
| 17 | 5-Methoxycarbonyl α-RhaRhaRha | C3 | 0.99 | 0.05 |
| 1 | 5-Methoxycarbonyl Anthrose | C4 | 0.97 | 0.03 |
| 35 | Pn23 polysaccharide | C5 | 18.50 | 1.48 |
| Subarray-f, IgG 10 μg/ml: | 5-Methoxycarbonyl Anthrose-β-Tetra 0.25 mg/ml | | | |
| 34 | Isolichenin | A1 | 10.40 | 0.43 |
| 21 | Me α-L-RhaGal | A2 | 1.09 | 0.02 |
| 24 | Me L-α-Rha((1→3)Rha | A3 | 1.05 | 0.04 |
| 25 | Me α-L-RhaRhaGal | A4 | 1.08 | 0.03 |
| 22 | Me α-D-GlcNAcRhaRhaGal | A5 | 1.05 | 0.01 |
| 3 | 5-Methoxycarbonyl Anthrose-α-Tri | B1 | 1.07 | 0.04 |
| 14 | 5-Methoxycarbonyl α-L-Rha | B2 | 0.98 | 0.01 |
| 16 | 5-Methoxycarbonyl αRha(1→3)Rha | B3 | 1.03 | 0.01 |
| 5 | 5-Methoxycarbonyl Anthrose-α-Tetra | B4 | 1.07 | 0.03 |
| 15 | 5-Methoxycarbonyl β-L-Rha | B5 | 1.06 | 0.05 |
| 4 | 5-Methoxycarbonyl Anthrose-β-Tetra | C1 | 1.04 | 0.06 |
| 20 | 5-Methoxycarbonyl β-RhaRhaRha | C2 | 1.06 | 0.05 |
| 17 | 5-Methoxycarbonyl α-RhaRhaRha | C3 | 1.04 | 0.02 |
| 1 | 5-Methoxycarbonyl Anthrose | C4 | 1.02 | 0.04 |
| 35 | Pn23 polysaccharide | C5 | 15.12 | 0.39 |

*IgG signal is measured as the ratio of the mean fluoresenct intensity of triplicate detections over the mean intensity of 112 blank spots in the same subarrays.
**See Supplementary Table 1 for structural information and references.

SUPPLEMENTARY TABLE 3
Glycan array characterization of rabbit polyclonal antibodies elicited by
B. anthracis spore immunization (Microarray data for FIG. 3)

| | |
|---|---|
| | *IgG signal |
| | detected by |
| | glycan |
| Saccharide arrays | arrays (n = 3) |

| Id# | **Short name | Mean | SD |
|---|---|---|---|
| 1 | 5-Methoxycarbonyl Anthrose | 1.44 | 0.08 |
| 2 | 5-Methoxycarbonyl Anthrose-α-Di | 1.65 | 0.06 |
| 3 | 5-Methoxycarbonyl Anthrose-α-Tri | 2.73 | 0.09 |
| 4 | 5-Methoxycarbonyl Anthrose-β-Tetra | 5.16 | 0.58 |
| 5 | 5-Methoxycarbonyl Anthrose-α-Tetra | 5.19 | 0.31 |
| 6 | L-Rha | 1.03 | 0.05 |
| 7 | D-Fuc | 1.88 | 0.21 |
| 8 | D-Gal | 1.06 | 0.05 |
| 9 | D-Glc | 1.04 | 0.00 |
| 10 | D-Man | 1.03 | 0.01 |
| 11 | GalNAC | 1.03 | 0.01 |
| 12 | L-Ara | 1.03 | 0.01 |
| 13 | L-Man | 1.06 | 0.04 |
| 14 | 5-Methoxycarbonyl α-L-Rha | 0.97 | 0.03 |
| 15 | 5-Methoxycarbonyl β-L-Rha | 0.95 | 0.01 |
| 16 | 5-Methoxycarbonyl α-Rha(1→3)Rha | 1.00 | 0.04 |
| 17 | 5-Methoxycarbonyl α-RhaRhaRha | 0.98 | 0.01 |
| 18 | 5-Methoxycarbonyl αRha(1→2)Rha | 1.26 | 0.07 |
| 19 | 5-Methoxycarbonyl βRha(1→2)Rha | 0.98 | 0.03 |
| 20 | 5-Methoxycarbonyl β-RhaRhaRha | 0.95 | 0.04 |
| 21 | Me α-L-RhaGal | 1.08 | 0.05 |
| 22 | Me α-D-GlcNAcRhaRhaGal | 0.95 | 0.03 |
| 23 | Me α-L-RhaGalGlcNAc | 1.01 | 0.02 |
| 24 | Me L-α-Rha(1→3)Rha | 1.02 | 0.04 |
| 25 | Me α-L-RhaRhaGal | 1.02 | 0.04 |
| 26 | Me α-L-RhaGalGlcNAc | 1.01 | 0.04 |
| 27 | Ogawa-Tri | 1.01 | 0.02 |
| 28 | Ogawa-Tetra | 1.07 | 0.02 |
| 29 | Ogawa-Pento | 1.09 | 0.06 |
| 30 | Ogawa-Hexa | 1.67 | 0.09 |
| 31 | Ogawa-Tri-BSA | 1.53 | 0.02 |
| 32 | Ogawa-Tetra-BSA | 1.20 | 0.04 |
| 33 | Ogawa-Pento-BSA | 1.37 | 0.05 |

*IgG signal is measured as the ratio of the mean fluoresenct intensity of triplicate detections over the mean intensity of 112 blank spots in the same subarrays.
**See Supplementary Table 1 for structural information and references.

### REFERENCES

[1] Mock, M., Fouet, A., Annu Rev Microbiol 2001, 55, 647-671.
[2] Webb, G. F., Proc Natl Acad Sci USA 2003, 100, 4355-4356.
[3] Newcombe, D. A., Schuerger, A. C., Benardini, J. N., Dickinson, D., et al., Appl Environ Microbiol 2005, 71, 8147-8156.
[4] Williams, D. D., Benedek, O., Turnbough, C. L., Jr., Appl Environ Microbiol 2003, 69, 6288-6293.
[5] Turnbull, P. C. B., Curr. Opin. Infect. Dis. 2000, 13, 113-120.
[6] Cohen, S., Mendelson, I., Altboum, Z., Kobiler, D., et al., Infect Immun 2000, 68, 4549-4558.
[7] Kramer, M. J., Roth, I. L., Can J Microbiol 1968, 14, 1297-1299.
[8] Kramer, M. J., Roth, I. L., Can J Microbiol 1969, 15, 1247-1248.
[9] Lai, E. M., Phadke, N. D., Kachman, M. T., Giorno, R., et al., J Bacteriol 2003, 185, 1443-1454.
[10] Redmond, C., Baillie, L. W., Hibbs, S., Moir, A. J., Moir, A., Microbiology 2004, 150, 355-363.
[11] Sylvestre, P., Couture-Tosi, E., Mock, M., Mol Microbiol 2002, 45, 169-178.
[12] Daubenspeck, J. M., Zeng, H., Chen, P., Dong, S., et al., J Biol Chem 2004, 279, 30945-30953.
[13] Steichen, C., Chen, P., Kearney, J. F., Turnbough, C. L., Jr., J Bacteriol 2003, 185, 1903-1910.
[14] Boydston, J. A., Chen, P., Steichen, C. T., Turnbough, C. L., Jr., J Bacteriol 2005, 187, 5310-5317.
[15] Carroll, G. T., Wang, D., Turro, N. J., Koberstein, J. T., Langmuir 2006, 22, 2899-2905.
[16] Saksena, R., Adamo, R., Kovac, P., Carbohydr Res 2005, 340, 1591-1600.
[17] Saksena, R., Adamo, R., Kovac, P., Bioorg Med Chem Lett 2006, 16, 615-617.
[18] Adamo, R., Saksena, R., Kovac, P., Carbohydr Res 2005, 340, 2579-2582.
[19] Wang, D., Liu, S., Trummer, B. J., Deng, C., Wang, A., Nat Biotechnol 2002, 20, 275-281.
[20] Wang, D., Lu, J., Physiol Genomics 2004, 18, 245-248.
[21] Wang, R., Liu, S., Shah, D., Wang, D., Methods Mol Biol 2005, 310, 241-252.
[22] Wang, D., Proteomics 2003, 3, 2167-2175.
[23] Chen, H. T., Kabat, E. A., J Biol Chem 1985, 260, 13208-13217.
[24] Pink, J. R. and Kiery, M. P., Vaccine 22: 2097-2102 (2004).
[25] Pashine, A. et al., Nature Medicine 11: S63-S68 (2005).
[26] Hoebe, K. et al., Nature Immunology 4: 1223-1229 (2003).

Second Series of Experiments

Anthrose-Containing Tetrasaccharide-PA Conjugate Displays Immunodominant Sugar Moiety

We have confirmed that a synthetic anthrose-containing tetrasaccharide the (α-isoforms)-conjugated with protective antigen (PA) of B. anthracis displays an antigenic determinant that highly specifically reacts with anti-anthrax spore IgG antibodies. This is evidenced by showing the relative contribution of various elements of the anthrose-containing tetrasaccharide to the specific anti-carbohydrate reactivities. For this purpose, we conducted enzyme-linked immunosorbent assay (ELISA)-based saccharide inhibition assays. On the ELISA microtiter plate, we applied a PA conjugate of α-anthrose-tetrasaccharide to display the saccharide moiety for antibody binding. Then, we examined the inhibitory activities of elements of this structure, including its terminal anthrose residue, internal Rha-trisaccharide chain, and two anomeric isoforms (α and β). As shown in FIG. 4B below, the two tetrasaccharides offer the stronger inhibition activities. The I⁵⁰ values for α- and β-isoforms are 0.18 and 0.23 nanomoles, respectively. The terminal anthrose is also an effective inhibitor with I⁵⁰ value of 7.3 nanomoles. By contrast, the internal chain trisaccharide structure gave no significant inhibition of the anti-anthrose-saccharide activities under the same experimental condition.

Anthrose-Containing Tetrasaccharide-α-PA Conjugate Elicits High Titer IgG Antibodies Specific for PA, as Well as a Number of Anthrose-Containing Saccharides and Rhamnose-Containing Saccharides

To validate whether the above saccharide-PA conjugate is able to elicit specific antibodies to both PA and the spore-saccharide determinants, we immunized a panel of six Balb/c mice using this conjugate. The initial immunization was conducted using 50 μg conjugate mixed with Complete Freund's Adjuvant (CFA) via i.p. injection. It was followed by two additional i.p. injections with the same amount of conjugate but mixed with Incomplete Freund's adjuvant (IFA). Sera were taken pre- and post-immunizations. Glycan array characterization of serum antibodies was conducted as described (Carroll, Wang et al. 2006; Wang, Carroll et al. 2006). All six of the immunized mice produced high titers of IgG and IgM antibodies for a number of anthrax spore-derived saccharide moieties. Importantly, this conjugate elicited production of antibodies for the protein carrier, PA. A representative glycan array result is shown in FIGS. 5A & 5B.

Methods

An ELISA-Based Saccharide Inhibition Assay (Chen and Rabat 1985)

ELISA microtiter plates (NUNC, MaxiSorp) were coated with a protein conjugate of α-anthrose-tetrasaccharide at 5 μg/ml in 0.1M NaBiCarbonate buffer, pH 9.6. The protein carrier is a preparation of recombinant Protective Antigen (rPA) of B. anthracis and was kindly provided by S. Leppla (NIAID, NIH). The rabbit anti-B. anthracis spore antibodies that were characterized in this study have no cross-reactivity with rPA. 100 μl of the conjugate were placed in each well; the wells with no antigen served as a blank. After 2 h incubation at 37° C., the wells were washed twice with PEST. The plates were blocked with 200 μl of 1% BSA PBST at room temperature for 1 h, and then washed twice. Varying quantities of the sugar inhibitors were mixed with a preparation of rabbit anti-anthrax spore IgG at 1:1000 dilutions and added to the antigen-coated ELISA plate; the wells with no inhibitor served as a standard. The total volume was adjusted to 100 μl per well with 1% BSA PEST and incubated at 37° C. for 1.5 h, then washed 5 times. 100 μl of an appropriately diluted biotinylated anti-rabbit IgG antibody (Sigma) were applied to reveal the bound rabbit IgG, which is followed by staining with Streptavidin-AP conjugate (Sigma) at 37° C. for 1 h. After washing 5 times, 100 μl of p-nitrophenyl phosphate solution, 30 mg/50 ml diethanolamine buffer, pH 9.8, were added and the reaction was placed at room temperature for 1 h. Twenty-five μl of 3 N NaOH were added to stop the reaction and the plates was read at 405 nm immediately. Percentage inhibition was calculated as follows: % inhibition=((standard A−blank A)−(A with inhibitor−blank A))/(standard A−blank A).

Method of Making Conjugates

All conjugates were made following the squaric acid chemistry conjugation protocol set forth below.

N denotes the number of sugar residues per carrier. Conjugates similar to those shown below were also made using bovine serum albumin (BSA). Structures of those conjugates are the same except that BSA replaces recombinant anthrax protective antigen (rPA).

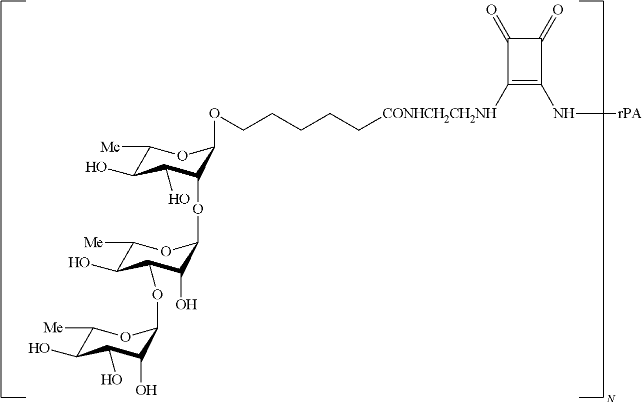

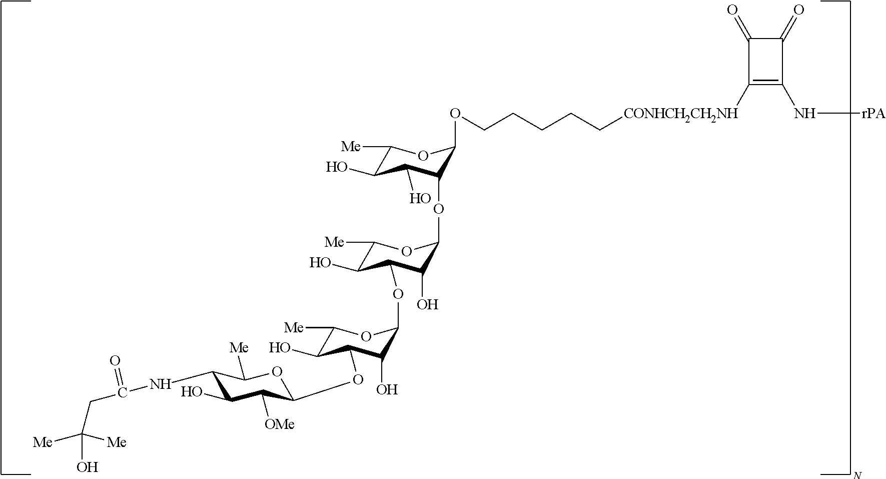

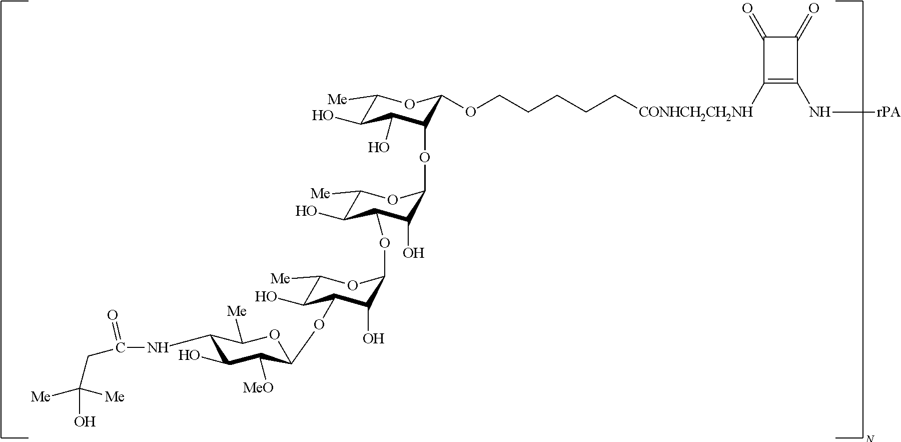

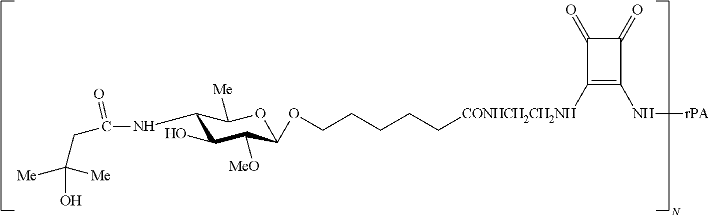

General Procedure for Conversion of Linker-Equipped Carbohydrates to their Corresponding Conjugates.

1. Conversion of linker-equipped carbohydrate (an ester) to its corresponding amide. A solution of the ester (0.1 mmol) and freshly distilled ethylenediamine (3 ml, 50 mmol) is heated at 50° C. until the reaction is complete (24-48 h). The reaction mixture is concentrated with the aid of oil pump and with co-evaporation of water and then chromatographed.

Purified material is dissolved in water, filtered through syringe filter and freeze-fried.

2. Conversion of amides to squaric acid derivatives. A solution of amine (0.02 mmol) and 3,4-diethoxy-3-cyclobutene-1,2-dione (2.5 eq., 0.05 mmol) in 1 ml of pH 7.00 buffer is stirred until the reaction is complete, as shown by TLC (3-24 h). The reaction mixture is concentrated, and the residue is chromatographed. A solution the pure compound in purified water is filtered through 0.22 μm sterile syringe filter and freeze-dried.

3. Conversion of squaric acid derivatives to neoglycoconjugates. The amount of the sugar and the carrier are calculated so that depending on the scale of the reaction the two reactant are present in 20:1 molar ratio. Carrier protein is added to borate buffer pH 9 in the amount to eventually form a 50 mM solution with respect to squaric acid derivative of the sugar, which is added after the carrier dissolves. The mixture is stirred at room temperature and the progress of conjugation is monitored by SELDI TOF Time-of-Flight Mass spectroscopy. When the desired carbohydrate ratio is reached, as determined by SELDI TOF Time-of-Flight Mass spectroscopy, the reaction is terminated by addition of borate buffer pH 7 (ten volumes of the original amount of the pH 9 buffer). The mixture is transferred into a centrifugal filter device and processed to remove salts. Freeze-drying affords the conjugate as a white solid.

General Procedure for Preparation of Amines from Esters (Saksena et al. (2003))

A solution of the ester (0.1 mmol) in ethylenediamine (20 mmol) was heated at 60-70° overnight, when TLC (1.3:1:0.1 CH₂Cl₂-MeOH-25% NH₄OH) showed that the reaction was complete. After concentration and evaporation of water from the residue (3 times), the solution of the crude product in water (˜2 mL) was applied on a SPE column (2-10 g sorbent mass, as required), which had been conditioned by washing with MeOH (30-80 mL), followed by water (50-100 mL). The elution was effected with water (20-50 mL), followed by a gradient of water (25 mL)→50% aq. MeOH (25 mL). Fractions of 2 mL were collected, analyzed by TLC, and those containing the desired material were concentrated to a small volume. Filtration through a syringe filter (0.45 μm porosity) and freeze-drying, gave products as white solids.

General Procedure for Preparation of Squaric Acid Monoesters (Saksena et al. (2003))

Diethyl squarate (0.015 mmol) was added to a solution of amines (0.01 mmol) in a potassium phosphate buffer pH 7.0 (3 mL), and the mixture was kept at room temperature overnight, when TLC (1:1 CH₂Cl₂-MeOH) showed that all the amine had been consumed and that a less polar, UV positive product was formed. Without any work-up, the mixture was subjected to SPE, using a column (sorbent mass, 5 g), which had been conditioned by washing with MeOH (40 mL), followed by water (60 mL). The elution was effected with water (20 mL), followed by a gradient of water (25 mL)→50% aq. MeOH (25 mL). Fractions of 2 mL were collected and analyzed by TLC. Fractions containing the desired material were concentrated and purified by preparative TLC, if necessary. An aqueous solution of the pure material, thus obtained, was filtered through a syringe filter (0.45 μm porosity) and freeze-dried, to give white or light-yellow solids.

General Procedure for the Preparation of Neoglycoconjugates (Saksena at al. (2003))

BSA (20 mg, 0.3 μmol) was suspended in a borate buffer, pH 9.0 (400 μL) and homogenized with the aid of a vortexer. The respective haptenic monoamide (0.0226 mmol) was transferred from its weighing container, using 1.1 mL of phosphate buffer pH 9.0 divided into three portions (400, 400 and 300 μL), into the above fine suspension of BSA. The clear solution thus formed was stirred at room temperature, while the progression of the conjugation was periodically monitored by SELDI-TOF MS, following the protocol recommended by Ciphergen for NP20 ProteinChips®. For monitoring the conjugation using BSA as the internal standard, a sample (1 μL) was withdrawn and mixed with pH 7 phosphate buffer (9 μL), giving solution A. 1 μL of a stock solution of BSA (20 mg/1.5 mL) was diluted with 9 μL of water, yielding solution B (internal standard). A portion of A (2 μL) was mixed with B (1 μL) and 1 μL of the resulting solution was applied on the ProteinChip®. The chip was air-dried, washed with water (5 μL, twice), with drying in between the washes and, finally, a solution (0.5 μL, twice) of the energy-absorbing molecule (satd solution of sinapinic acid in 1% TFA) was applied. The chip was air-dried, followed by reading the molecular masses. Two peaks corresponding to single-charged molecules were observed. The average hapten/BSA ratio was calculated from the difference between the molecular masses shown for BSA and the glycoconjugate formed. When the required hapten/BSA ratio was reached, 500 □L of the solution was withdrawn, diluted with phosphate buffer pH 7.0 (3 mL), and subjected to ultra filtration using the Amicon cell equipped with PM-10 membrane (Millipore Copporation, Bedford, Mass.). The retained material was freeze-dried, to give conjugates in 80-95% yields. The molecular mass of the final products was verified by SELDI-TOF MS.

### REFERENCES

Carroll, G. T., D. Wang, et al. (2006). “Photochemical micropatterning of carbohydrates on a surface.” Langmuir 22(6): 2899-905.
Chen, H. T. and E. A. Kabat (1985). “Immunochemical studies on blood groups. The combining site specificities of mouse monoclonal hybridoma anti-A and anti-B.” J Biol Chem 260(24): 13208-17.
Saksena, R. et al. (2003) Carbohydrate Research 338: 2591-2603.

Third Series of Experiments

Anthrose-Containing Tetrasaccharide-PA Conjugates Display Two Antigenic Determinants: Anthrose-Terminal Epitopes and Rha2/3-Internal Epitopes

Synopsis

Presented here is a high-throughput strategy for identification of immunogenic sugar moieties of microbial pathogens and characterization of antibody responses to carbohydrate antigens. Using photo-generated gylcan arrays, a large panel of synthetic carbohydrates was characterized for their antigenic reactivities with pathogen-specific antibodies. It was found that rabbit IgG antibodies elicited by B. anthracis spores recognize the sugar moieties that decorate the outermost surfaces of B. anthracis exosporium. The saccharide binding reactivities correlate directly with the size of the saccharides displayed by the glycan arrays. The terminal non-reducing end sugar residue, anthrose, is marginally reactive and the anthrose-containing tetrasaccharide highly reactive. Mice immunized with a conjugate of this tetrasaccharide and the protective antigen (PA) of B. anthracis produced high titers of serum IgG antibodies for the conjugate. Glycan array analysis of the serum antibodies further demonstrates that there is presence of specificities for the saccharide moieties and specificities for the protein carrier PA, respectively. Thus, both the native antigens of the B. anthracis spore and the synthetic glycoconjugate elicited IgG antibody responses to the anthrose-containing tetrasaccharide. Therefore, a potent immunogenic carbohydrate moiety of B. anthracis was identified. Since this antigenic structure is highly specific for the spores of B. anthracis, it is a key immunological marker for anthrax spore identification.

Experimental Approach

A group of six Balb/c mice were immunized with a Rha-trisaccharide-PA conjugate. Glycan array characterization of serum antibodies was conducted. All six of the mice elicited high titers of IgG antibodies for the conjugate, including anti-carbohydrate specificities and anti-PA specificities.

A group of five SLJ/J mice were immunized with anthrose-tetrasaccharide-BSA3.5 (molar ratio of saccharide and BSA in the conjugate is 3.5). Glycan array characterization of serum antibodies was conducted. The immunization elicited high titers of IgG anti-anthrose-tetrasaccharide antibodies.

A group of five SLJ/J mice were immunized with antrhose-tetrasaccharide-BSA5.7 (molar ratio of saccharide and BSA in the conjugate is 5.7). Glycan array characterization of serum antibodies was conducted. The immunization elicited high titers of IgG anti-anthrose-tetrasaccharide antibodies.

The anti-glycan antibody specificities demonstrated above were reproduced by antigen-specific enzyme-linked immunosorbent assay (ELISA) binding and saccharide competition assays.

A microarray-based semi-quantitative analysis of antigen-specific antibodies in Balb/c serum specimens was performed pre- and post-immunization. Sera from ten Balb/c mice pre- and post-immunization with Anthrose-tetrasaccharide-PA conjugates or Rha-trisaccharide-PA conjugates were characterized. The mean IgG values (Int-Bg) at pre-immunization day zero were compared to the mean IgG values (Int-Bg) at immunization day 21 for the group of mice immunized with anthrose-tetrasaccharide-PA conjugate and Rha-trisaccharide-PA conjugate as shown in FIG. 6. The microarray datasets were processed and statistically analyzed using SAS Institute's MP 6.0 software package.

Results

The results of these experiments reveal two antigenic determinants of the anthrose-tetrasaccharide of B. anthracis spores comprising a terminal non-reducing end epitope, which is composed of the terminal anthrose and two adjacent rhamnose residues with α(1,3) linkage, and an internal sugar epitope, which is formed by α(1,3) linked rhamnose (Rha) disaccharide or trisaccharide (See FIG. 7). The dominant antigenic determinant in both rabbit and mouse is the anthrose-containing terminal epitope which is able to elicit highly specific antibody response to B. anthracis spores. The internal Rha-epitope is a potent antigenic determinant in mouse but not in rabbit so far studied. Since a number of human pathogens express Rha-containing sugar moieties and these moieties are usually highly immunogenic, the Rha-containing internal epitope may elicit immune responses that are cross reactive with these pathogens and thus offer a broad-range of protection to microbial infection.

1-14. (canceled)

15. A method for vaccinating a subject against Bacillus anthracis infection caused by the inhalation of Bacillus anthracis spores, comprising administering to the subject a vaccine comprising (i) a conjugate of an anthrose-containing saccharide in an amount effective to enhance immunity against Bacillus anthracis in a subject, wherein the anthrose-containing saccharide is conjugated to a biomolecule via a linker, and (ii) a pharmaceutically acceptable carrier, in an amount effective to stimulate production of antibodies to Bacillus anthracis spores in the subject, thereby vaccinating the subject against Bacillus anthracis infection caused by the inhalation of Bacillus anthracis spores.

16. An isolated antibody which specifically binds to an epitope on an anthrose-containing saccharide or an isolated anti-idiotypic antibody which specifically binds to the antibody which specifically binds to an anthrose-containing saccharide.

17-25. (canceled)

26. A composition of matter comprising (i) an antibody which specifically binds to an epitope on an anthrose-containing saccharide, and (ii) a detectable marker, wherein the detectable marker is conjugated to the antibody.

27. (canceled)

28. A composition of matter comprising (i) an antibody which specifically binds to an epitope on an anthrose-containing saccharide and (ii) an antibiotic against Bacillus anthracis, wherein the antibiotic against Bacillus anthracis is conjugated to the antibody.

29-32. (canceled)

33. A composition comprising (i) an antibody which specifically binds to an anthrose-containing saccharide in an amount effective to enhance immunity against Bacillus anthracis in a subject and (ii) a pharmaceutically acceptable carrier.

34. A method for reducing the likelihood of a subject being infected with Bacillus anthracis, which comprises administering to the subject a prophylactically effective amount of a composition comprising (i) an antibody which specifically binds to an anthrose-containing saccharide, and (ii) a pharmaceutically acceptable carrier, thereby reducing the likelihood of a subject being infected with Bacillus anthracis.

35-37. (canceled)

38. A method for determining whether Bacillus anthracis spores are present in a serum sample of a subject which comprises:
(a) contacting the serum sample with an antibody which specifically binds to an epitope on an anthrose-containing saccharide under conditions that permit the formation of a complex between the antibody and an anthrose-containing saccharide; and
(b) detecting the presence of the complex formed in step (a);
wherein the presence of such complex indicates that Bacillus anthracis spores are present in the serum sample.

39. A method for determining whether a subject has been exposed to Bacillus anthracis which comprises:
(a) contacting a serum sample from the subject with an antibody which specifically binds to an epitope on an anthrose-containing saccharide under conditions that permit the formation of a complex between the antibody and an anthrose-containing saccharide; and
(b) detecting the presence of the complex formed in step (a);
wherein the presence of such complex indicates that the subject has been exposed to Bacillus anthracis.

40. A method for determining whether a subject is infected with Bacillus anthracis which comprises:
(a) contacting a serum sample from the subject with a anti-idiotypic antibody which specifically binds to an antibody which specifically binds to an anthrose-containing saccharide under conditions which permit the formation of a complex between the anti-idiotypic antibody and an antibody which specifically binds to an anthrose-containing saccharide;
(b) determining the amount of complex formed in step (a); and
(c) comparing the amount of complex determined in step (b) with an amount of complex correlative with that found for a subject infected with Bacillus anthracis,
thereby determining whether the subject is infected with Bacillus anthracis.

41. A kit for detecting the presence of Bacillus anthracis spores in a sample comprising, in separate compartments, (i) an antibody which specifically binds to an epitope of an anthrose-containing saccharide, and (ii) reagents for detecting binding of the antibody to an anthrose-containing saccharide.

42-52. (canceled)

53. The method of claim 15, wherein the biomolecule is a polypeptide.

54. The method of claim 53, wherein the polypeptide is protective antigen (PA) of Bacillus anthracis.

55. The method of claim 53, wherein the polypeptide is bovine serum albumin (BSA).

56. The method of claim 15, wherein the linker comprises a squaric acid derivative.

57. The method of claim 15, wherein the vaccine further comprises (iii) a pharmaceutically acceptable adjuvant.

58. The method of claim 15, wherein the anthrose-containing saccharide is a monosaccharide, disaccharide, trisaccharide, or tetrasaccharide.

59. The method of claim 15, wherein the anthrose-containing saccharide is a tetrasaccharide.

60. The method of claim 59, wherein the tetrasaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoyl-(1→3)-α-L-rhamnopyranosyl-(1→2)-β-L-rhamnopyranoside.

61. The method of claim 59, wherein the tetrasaccharide is 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoyl-(1→3)-α-L-rhamnopyranosyl-(1→2)-α-L-rhamnopyranoside.

62. The method of claim 58, wherein the anthrose-containing saccharide is 5-(Methoxycarbonyl) pentyl 4-(3-hydroxy-3-methylbutanamido)-2-O-methyl-4,6-dideoxy-β-D-glucopyranosyl-(1→3)-α-L-rhamnopyranoside or 5-(Methoxycarbonyl)pentyl 4-(3-hydroxy-3-methylbutanamido)-2-O-methyl-4,6-dideoxy-β-D-glucopyranosyl-(1→3)-α-L-rhamnopyranoyl-(1→3)-α-L-rhamnopyranosyl-(1→2)-β-L-rhamnopyranoside.

63. The method of claim 60, wherein the subject is a mammal.

64. The method of claim 63, wherein the mammal is a human.

65. The method of claim 63, wherein vaccinating the subject is effective to slow the progression of a Bacillus anthracis infection caused by the inhalation of Bacillus anthracis spores.

66. The method of claim 63, wherein vaccinating the subject is effective to prevent a Bacillus anthracis infection caused by the inhalation of Bacillus anthracis spores.

67. The method of claim 65, wherein the amount of the vaccine that is administered to the subject is 10 μg/kg-1 mg/kg.

68. The method of claim 67, wherein the amount of the vaccine that is administered to the subject is 10-200 μg/kg.
